(19) United States
(12) Patent Application Publication
(10) Pub. No.: US 2016/0101176 A1
(43) Pub. Date: Apr. 14, 2016

(54) POLYMERIC HYDROGEL PHARMACEUTICAL COMPOSITIONS WITH ON-DEMAND RELEASE OF A DRUG IN RESPONSE TO A ELECTRICAL STIMULUS

(71) Applicant: UNIVERSITY OF THE WITWATERSRAND, JOHANNESBURG, Johannesburg (ZA)

(72) Inventors: Viness PILLAY, Sandton (ZA); Sunaina INDERMUN, Durban (ZA); Lisa Claire DU TOIT, Florida (ZA); Yahya Essop CHOONARA,, Johannesburg (ZA); Pradeep KUMAR, Johannesburg (ZA)

(73) Assignee: UNIVERSITY OF THE WITWATERSRAND, JOHANNESBURG, Johannesburg (ZA)

(21) Appl. No.: 14/894,144

(22) PCT Filed: Jun. 2, 2014
(86) PCT No.: PCT/IB2014/061890

§ 371 Date: Nov. 25, 2015

(30) Foreign Application Priority Data
May 31, 2013 (ZA) 2013/03983

(51) Int. Cl. A61K 41/00; A61K 9/06; A61K 31/405; A61K 47/32; A61K 47/34; A61K 47/22

(52) U.S. Cl. CPC A61K 41/0023; A61K 47/34; A61K 47/22; A61K 31/405; A61K 47/32; A61K 9/06

(57) ABSTRACT

A polymeric hydrogel pharmaceutical dosage form for drug delivery to a target site of a human or animal. The dosage form includes polyethylene-imine (PEI) and 1-vinylimidazole (1VA), the dosage form being electro-responsive in use. Also, methods of manufacturing the dosage form and methods of treating chronic pain utilizing the dosage form.

### FIELD

The invention relates to a pharmaceutical dosage form containing a drug and capable of drug release via stimulus activation from an external device. Particularly, the invention relates to a polymeric hydrogel pharmaceutical dosage form capable of drug release when an electric current is applied to the hydrogel.

### BACKGROUND

There has been a significant amount of research directed toward pharmaceutical dosage forms including oral and intravenous dosage forms. The main disadvantage of oral dosage forms is the hepatic first pass metabolism of the drug to be delivered by the dosage form and also gastrointestinal degradation. The main disadvantage of intravenous dosage forms is the pain and phobia associated with needles necessary for intravenous administration of the dosage form containing the drug.

Consequently, there is a need for developing alternative parenteral dosage forms which show a high degree of patient compliance and are effective in ensuring drug delivery to a specific target site.

In regard to the particular type of drug to be administered there is a major need to develop dosage forms for the fast and efficient delivery of analgesics. Pain management, and in particular chronic pain management, has always been challenging for both clinician and patient. Chronic intravenous administration causes damage to the dermis of the patient and creates a new source of pain. Chronic oral administration may include a host of side effects depending on the formulation of the oral dosage form, including for example the formation of gastric ulcers. Oral dosage forms may also take a fair amount of time to provide effective pain relief since the dosage form will need to dissolve and release the analgesic drug in certain areas of the gastrointestinal tract before a patient experiences pain relief.

Transdermal dosage forms have been suggested as a patient compliant parenteral dosage form alternative for chronic pain management. There are many challenges in providing a transdermal dosage form allowing for long term application to the dermis of a patient and also allowing for patient modulated drug release to manage chronic pain as needed by the patient.

### SUMMARY

According to a first aspect of this invention there is provided a polymeric hydrogel pharmaceutical dosage form for drug delivery to a target site of a human or animal, the dosage form comprising:

polyethyleneimine (PEI) and 1-vinylimidazole (1VA),

wherein application of an electrical stimulus to the dosage form induces a first conformational change in the dosage form resulting in a release conformation which facilitates an increase in the release rate of the drug from the dosage form to the target site, and
wherein cessation of the electrical stimulus to the dosage form induces a second conformational change in the dosage form resulting in a drug containing conformation which facilitates a decrease in the release rate of the drug from the dosage form to the target site.

Preferably, in use, cessation of the electrical stimulus causes cessation of the release of the drug from the dosage form to the target site.

The dosage form may further comprise polyacrylic acid (PAA) and/or polyvinyl alcohol (PVA).

The dosage form may further comprise a crosslinking agent, preferably N,N′-methylenebisacrylamide.

The dosage form may further comprise a crosslinking initiator, preferably potassium persulfate.

The crosslinking agent may in use crosslink at least one or more of the following group: polyacrylamide (PAA), polyethyleneimine (PEI), polyvinyl alcohol (PVA) and 1-vinylimidazole (1VA).

The target site may be the dermis of the human or animal.

The dosage form may further include at least one drug. Typically, the dosage form may be for use in relieving or ameliorating chronic pain, and the drug may be an analgesic, and is preferably a non-steroidal anti-inflammatory drug (NSAID) such as indomethacin. The drug may for example also be morphine, celecoxib and/or fentanyl chloride.

The electrical stimulus may be an electric current. The electric current may be applied to the dosage form from about 0.1 seconds to about 60 seconds, and any points in between. The electric current may have a voltage of from about 0.3 volts to about 5 volts, and any points in between.

When the dosage form is in the drug release conformation the release rate of the drug from the dosage form to the target site via diffusion is increased.

When the dosage form is in the drug containing conformation the release rate of the drug from the dosage form to the target site via diffusion is decreased and may cease.

In use, the polyethyleneimine (PEI) may be electro-conductive allowing for conduction of the electrical stimulus therethrough. Further in use, the polyethyleneimine may be electro-responsive such that application of an electrical stimulus induces a structural change in the polyethyleneimine (PEI).

In use, the 1-vinylimidazole (1VA) may be electro-conductive allowing for conduction of the electrical stimulus therethrough. Further in use, the 1-vinylimidazole may be electro-responsive such that application of an electrical stimulus induces a structural change in the 1-vinylimidazole (1VA). Still further in use, the 1-vinylimidazole (1VA) may be a plasticizer so as to increase the plasticity and/or fluidity of the dosage form in use.

In use, the polyvinyl alcohol (PVA) may provide mechanical strength and/or robustness.

In use, the polyacrylic acid (PAA) may be electro-conductive allowing for conduction of the electrical stimulus therethrough.

The Applicant has noticed that known hydrogels including individually either polyvinyl alcohol (PVA) or polyacrylic acid (PAA) result in hydrogels that show poor viscosity and undesirably high brittleness respectively. Consequently, the hydrogel pharmaceutical dosage form according to the first aspect of the invention, which shows desirable mechanical strength and/or robustness and desirable viscosity in use, was wholly unexpected and surprising.

The Applicant is not aware of 1-vinylimidazole (1VA) forming part of known hydrogel pharmaceutical dosage forms let alone how 1-vinylimidazole (1VA) would interact with polyethyleneimine (PEI) to form a polymeric hydrogel pharmaceutical dosage form wherein application of the electrical stimulus to the dosage form induces the first conformational change in the dosage form resulting in the release conformation which facilitates an increase in the release rate of the drug from the dosage form to the target site, and wherein cessation of the electrical stimulus to the dosage form induces the second conformational change in the dosage form resulting in the drug containing conformation which facilitates a decrease in the release rate of the drug from the dosage form to the target site.

According to a second aspect of this invention there is provided a polymeric hydrogel pharmaceutical dosage form for drug delivery to a target site of a human or animal, the dosage form comprising:

polyethyleneimine (PEI) and 1-vinylimidazole (1VA) forming an electro responsive matrix;

polyacrylic acid (PAA) and/or polyvinyl alcohol (PVA) at least partially crosslinked with the matrix and at least partially penetrating the matrix to form an interpenetrating polymer network,

wherein application of an electrical stimulus to the dosage form induces a first conformational change in the interpenetrating polymer network resulting in a release conformation which facilitates an increase in the release rate of the drug from the dosage form to the target site, and
wherein cessation of the electrical stimulus to the dosage form induces a second conformational change in the interpenetrating polymer network resulting in a drug containing conformation which facilitates a decrease in the release rate of the drug from the dosage form to the target site.

Preferably, in use, cessation of the electrical stimulus causes cessation in the release of the drug from the dosage form to the target site.

The dosage form may further comprise a crosslinking agent, preferably N,N′-methylenebisacrylamide.

The dosage form may further comprise a crosslinking initiator, preferably potassium persulfate.

The crosslinking agent may in use crosslink at least one or more of the following group: polyacrylamide (PAA), polyethyleneimine (PEI), polyvinyl alcohol (PVA) and 1-vinylimidazole (1VA).

The target site may be the dermis of the human or animal.

The dosage form may further include at least one drug. Typically, the dosage form may be for use in relieving or ameliorating chronic pain, and the drug may be an analgesic, and is preferably a non-steroidal anti-inflammatory drug (NSAID) such as indomethacin. The drug may for example also be morphine, celecoxib and/or fentanyl chloride.

The electrical stimulus may be an electric current. The electric current may be applied to the dosage form from about 0.1 seconds to about 60 seconds, and any points in between. The electric current may have a voltage of from about 0.3 volts to about 5 volts, and any points in between.

When the dosage form is in the drug release conformation the release rate of the drug from the dosage form to the target site via diffusion is increased.

When the dosage form is in the drug containing conformation the release rate of the drug from the dosage form to the target site via diffusion is decreased and may cease.

In use, the polyethyleneimine (PEI) may be electro-conductive allowing for conduction of the electrical stimulus therethrough. Further in use, the polyethyleneimine may be electro-responsive such that application of an electrical stimulus induces a structural change in the polyethyleneimine (PEI).

In use, the 1-vinylimidazole (1VA) may be electro-conductive allowing for conduction of the electrical stimulus therethrough. Further in use, the 1-vinylimidazole may be electro-responsive such that application of an electrical stimulus induces a structural change in the 1-vinylimidazole (1VA). Still further in use, the 1-vinylimidazole (1VA) may be a plasticizer so as to increase the plasticity and/or fluidity of the dosage form in use.

In use, the polyvinyl alcohol (PVA) may provide mechanical strength and/or robustness.

In use, the polyacrylic acid (PAA) may be electro-conductive allowing for conduction of the electrical stimulus therethrough.

The Applicant has noticed that known hydrogels including individually either polyvinyl alcohol (PVA) or polyacrylic acid (PAA) result in hydrogels that show poor viscosity and undesirably high brittleness respectively. Consequently, the hydrogel pharmaceutical dosage form according to the second aspect of the invention, which shows desirable mechanical strength and/or robustness and desirable viscosity in use, was wholly unexpected and surprising.

The Applicant is not aware of 1-vinylimidazole (1VA) forming part of known hydrogel pharmaceutical dosage forms let alone a hydrogel dosage form including 1-vinylimidazole (1VA) and polyethyleneimine (PEI) forming a matrix, and further including polyacrylic acid (PAA) and/or polyvinyl alcohol (PVA) each of which at least partially crosslinks with the matrix and at least partially penetrates the matrix to form an interpenetrating polymer network polymeric hydrogel pharmaceutical dosage form, wherein application of the electrical stimulus to the dosage form induces the first conformational change in the dosage form resulting in the release conformation which facilitates an increase in the release rate of the drug from the dosage form to the target site, and wherein cessation of the electrical stimulus to the dosage form induces the second conformational change in the dosage form resulting in the drug containing conformation which facilitates a decrease in the release rate of the drug from the dosage form to the target site.

The dosage form according to the first or second aspect of the invention may form part of a system for transdermal drug delivery, for example, a skin patch. In a preferred embodiment of the invention, the system for transdermal drug delivery is a microneedle array skin patch assembly.

According to a third aspect of the invention there is provided a method of manufacturing a polymeric hydrogel pharmaceutical dosage form for drug delivery to a target site of a human or animal, the method comprising the following step(s):
(a) mixing together polyethyleneimine (PEI) and 1-vinylimidazole (1VA) to form a first solution;
(b) adding polyvinyl alcohol (PVA) and acrylic acid (AA) to the first solution to form a second solution; and
(c) allowing a polymeric hydrogel to form.

The method according to the third aspect of the invention may comprise an additional step (d), wherein step (d) includes adding a drug to the first solution in order to manufacture a drug loaded polymeric hydrogel pharmaceutical dosage form.

The method may further comprise step (e), wherein step (e) includes adding a crosslinking agent to the second solution, preferably the crosslinking agent may be N,N′-methylenebisacrylamide.

The method may further comprise step (f), wherein step (f) includes adding crosslinking initiator to the second solution, preferably the crosslinking initiator is potassium persulfate.

The polymeric hydrogel pharmaceutical dosage form may be that according to the first aspect of the invention.

According to a fourth aspect of the invention there is provided a method of manufacturing a polymeric hydrogel pharmaceutical dosage form for drug delivery to a target site of a human or animal, the method comprising the following step(s):
(a) mixing together polyethyleneimine (PEI), 1-vinylimidazole (1VA) and a drug to form a first solution;
(b) adding polyvinyl alcohol (PVA) and acrylic acid (AA) to the first solution to form a second solution
(c) allowing a polymeric hydrogel to form which contains the drug and is responsive to electrical stimulus.

The method may further comprise step (d), wherein step (d) includes adding a crosslinking agent to the second solution, preferably the crosslinking agent may be N,N′-methylenebisacrylamide.

The method may further comprise step (e), wherein step (e) includes adding crosslinking initiator to the second solution, preferably the crosslinking initiator is potassium persulfate.

According to a fifth aspect of the invention there is provided a method of manufacturing a polymeric hydrogel pharmaceutical dosage form for drug delivery to a target site of a human or animal, the method comprising the following step(s):
(a) preparing a polyvinyl alcohol (PVA) solution to which polyethyleneimine (PEI) and 1-vinylimidazole (1VA) is added to form a first mixture;
(b) adding a drug, acrylic acid and a crosslinking agent to the first mixture; and
(c) allowing a hydrogel to form which contains the drug and is responsive to electrical stimulus.

According to a sixth aspect of the invention there is provided for a method of treating chronic pain in a human or animal, the method comprising the steps of:

applying the polymeric hydrogel pharmaceutical dosage form according to the first and/or second aspect of the invention to a target site for drug delivery; and

applying an electrical stimulus to the dosage form wherein application of the electrical stimulus to the dosage form induces a first conformational change in the dosage form resulting in a release conformation which facilitates an increase in the release rate of the drug from the dosage form to the target site, and wherein cessation of the electrical stimulus to the dosage form induces a second conformational change in the dosage form resulting in a drug containing conformation which facilitates a decrease in the release rate of the drug from the dosage form to the target site. Preferably, in use, cessation of the electrical stimulus causes cessation in the release of the drug from the dosage form to the target site.

There is provided for the polymeric hydrogel pharmaceutical dosage form, methods to manufacture the same and methods of treatment as substantially described, illustrated and/or exemplified herein with reference to any one of the drawings and/or examples and/or tables.

### BRIEF DESCRIPTION OF THE DRAWINGS

FIG. 1 shows a schematic representation of PEiGOR theory applied to a hydrogel according to the invention wherein only polyethyleneimine (PEI), polyacrylic acid (PAA) and an example drug are shown for the sake of simplicity, and wherein frame (a) shows the hydrogel prior to electrical stimuli (b) shows the hydrogel during electrical stimulation showing the drug release conformation and (c) shows the hydrogel after electrical stimuli showing the drug containing conformation;

FIG. 2a shows an energy plot of geometrical optimization mapping over a number of iteration cycles for a PEI-PAA₂-1VA₄-H₂O (0.1 a.u. in direction x of the three dimensional simulated structure) hydrogel resulting from molecular simulations in a solvated system under external electric field;

FIG. 2b shows an energy plot of geometrical optimization mapping over a number of iteration cycles for a PEI-PAA₂-1VA₄-H₂O (0.3 a.u. in direction x of the three dimensional simulated structure) hydrogel resulting from molecular simulations in a solvated system under external electric field;

FIG. 2c shows an energy plot of geometrical optimization mapping over a number of iteration cycles for a PEI-PAA₂-1VA₄-H₂O (0.5 a.u. in direction x of the three dimensional simulated structure) hydrogel resulting from molecular simulations in a solvated system under external electric field;

FIG. 3a shows an energy plot of geometrical optimization mapping over a number of iteration cycles for a PEI-PAA₂-1VA₄-H₂O (0.1 a.u. in direction y of the three dimensional simulated structure) hydrogel resulting from molecular simulations in a solvated system under external electric field;

FIG. 3b shows an energy plot of geometrical optimization mapping over a number of iteration cycles for a PEI-PAA₂-1VA₄-H₂O (0.3 a.u. in direction y of the three dimensional simulated structure) hydrogel resulting from molecular simulations in a solvated system under external electric field;

FIG. 3c shows an energy plot of geometrical optimization mapping over a number of iteration cycles for a PEI-PAA2-1VA₄-H₂O (0.5 a.u. in direction y of the three dimensional simulated structure) hydrogel resulting from molecular simulations in a solvated system under external electric field;

FIG. 4a shows an energy plot of geometrical optimization mapping over a number of iteration cycles for a PEI-PAA₂-1VA₄-H₂O (0.1 a.u. in direction z of the three dimensional simulated structure) hydrogel resulting from molecular simulations in a solvated system under external electric field;

FIG. 4b shows an energy plot of geometrical optimization mapping over a number of iteration cycles for a PEI-PAA₂-1VA₄-H₂O (0.3 a.u. in direction z of the three dimensional simulated structure) hydrogel resulting from molecular simulations in a solvated system under external electric field;

FIG. 4c shows energy plot of geometrical optimization mapping over a number of iteration cycles for a PEI-PAA₂-1VA₄-H₂O (0.5 a.u. in direction z of the three dimensional simulated structure) hydrogel resulting from molecular simulations in a solvated system under external electric field;

FIG. 5 shows an energy plot of geometrical optimization mapping over a number of iteration cycles for a PEI-PAA₂-1VA₄-H₂O hydrogel resulting from molecular simulations in a solvated system under no external electric field;

FIG. 6 shows drug release profiles indicating the effect of lyophilization on the optimized hydrogel formulation (in all cases N=3; SD≦0.34);

FIG. 7a shows drug release profiles of Box-Behnken design Formulations 1-5 with indomethacin as the example drug;

FIG. 7b shows drug release profiles of Box-Behnken design Formulations 6-10 with indomethacin as the example drug;

FIG. 7c shows drug release profiles of Box-Behnken design Formulations 11-15 with indomethacin as the example drug;

FIG. 8 shows desirability plots representing the levels of polyethyleneimine (PEI), 1-vinylimidazole (1VA) and voltage required to synthesize the optimized formulation;

FIG. 9 shows drug release profile of the Optimized Formulation containing Indomethacin as an example drug; 3 optimized formulations were tested to ensure reproducibility;

FIG. 10a shows a drug release profile of the Optimized Formulation containing of morphine HCL as the example drug;

FIG. 10b shows a drug release profile of the Optimized Formulation containing of celecoxib as the example drug;

FIG. 10c shows a drug release profile of the Optimized Formulation containing of fentanyl citrate as the example drug;

FIG. 11 shows an example embodiment of a system for transdermal drug delivery including the polymeric hydrogel pharmaceutical dosage form according to the first and/or second aspects of the invention;

FIG. 12 shows a schematic diagram showing the design of the in vivo animal studies utilizing the system illustrated in FIG. 11;

FIG. 13 shows in vivo drug concentrations attained from the conventional and experimental groups (SD≦2.55×10⁻⁶; n=6) of the animal studies;

FIG. 14 shows drug release profiles of the in vitro release and the observed mean in vivo release profile extracted using deconvolution analysis of indomethacin from the system of FIG. 11 as used in the animal studies;

FIG. 15 shows a regression plot showing the relationship between the fraction of indomethacin absorbed in vivo and the fraction released in vitro; and

FIG. 16 shows drug release profiles of the observed and predicted in vitro indomethacin release.

### DETAILED DESCRIPTION

According to a first aspect of this invention there is provided a polymeric hydrogel pharmaceutical dosage form for drug delivery to a target site of a human or animal, the dosage form comprising polyethyleneimine (PEI) and 1-vinylimidazole (1VA). In use, application of an electrical stimulus to the dosage form induces a first conformational change in the dosage form resulting in a release conformation which facilitates an increase in the release rate of the drug from the dosage form to the target site, and wherein cessation of the electrical stimulus to the dosage form induces a second conformational change in the dosage form resulting in a drug containing conformation which facilitates which facilitates a decrease in the release rate of the drug from the dosage form to the target site. Preferably, in use, cessation of the electrical stimulus causes cessation in the release of the drug from the dosage form to the target site. The target site is usually the dermis of the human or animal body, however, it is to be understood that the target site may be other sites on or in the human or animal body.

The dosage form is a polymeric hydrogel. Hydrogels are known in the art and are often, but not exclusively, a substance formed when an organic polymer (natural or synthetic) is cross-linked via covalent, ionic, or hydrogen bonds to create a three-dimensional open-lattice structure which entraps water molecules to form a gel.

The dosage form typically further comprises polyacrylic acid (PAA) and/or polyvinyl alcohol (PVA) and/or a crosslinking agent, preferably the crosslinking agent is N,N′-methylenebisacrylamide. The crosslinking agent may in use crosslink at least one or more of the following group: polyacrylamide (PAA), polyethyleneimine (PEI), polyvinyl alcohol (PVA) and 1-vinylimidazole (1VA).

In a preferred embodiment of the invention the dosage form further comprises a crosslinking initiator, preferably in the form of potassium persulfate.

The dosage form may be a placebo and therefore lack a drug compound, alternatively, the dosage form may be drug loaded and contain a drug compound. Generally, the dosage form is drug loaded. Although it is envisioned that the dosage form could be used to treat a range of medical conditions and/or diseases, typically the dosage form is for use in relieving or ameliorating chronic pain, and the drug may be an analgesic, and is preferably a non-steroidal anti-inflammatory drug (NSAID) such as but not limited to indomethacin. The drug may for example also be morphine, celecoxib and/or fentanyl chloride.

The drug containing conformation slows the release of the drug relative to when the electrical stimulus is applied and may slow drug release to the point where no drug is released whatsoever. Typically, the electrical stimulus increases the rate of diffusion of the drug to the target site. Generally speaking, and as described, illustrated and/or exemplified hereunder in more detail the electrical stimulus is an electric current. The electric current may be applied to the dosage form from about 0.1 seconds to about 60 and any points in between. The electric current may have a voltage of from about 0.3 volts to about 5 volts, and any points in between.

Each component of the dosage form has particular physico-chemical and/or physico-mechanical properties.

In use, the polyethyleneimine (PEI) is electro-conductive allowing for conduction of the electrical stimulus therethrough. Further in use, the polyethyleneimine is electro-responsive such that application of an electrical stimulus induces a structural change in the polyethyleneimine (PEI).

In use, the 1-vinylimidazole (1VA) is electro-conductive allowing for conduction of the electrical stimulus therethrough. Further in use, the 1-vinylimidazole is electro-responsive such that application of an electrical stimulus induces a structural change in the 1-vinylimidazole (1VA). Still further in use, the 1-vinylimidazole (1VA) is a plasticizer so as to increase the plasticity and/or fluidity of the dosage form in use.

In use, the polyvinyl alcohol (PVA) provides mechanical strength and/or robustness.

In use, the polyacrylic acid (PAA) is electro-conductive allowing for conduction of the electrical stimulus therethrough.

The Applicant has noticed that known hydrogels including individually either polyvinyl alcohol (PVA) or polyacrylic acid (PAA) result in hydrogels that show poor viscosity and undesirably high brittleness respectively. Consequently, the hydrogel pharmaceutical dosage form according to the first aspect of the invention, which shows desirable mechanical strength and/or robustness and desirable viscosity in use, was wholly unexpected and surprising. The dosage form according to the invention is robust enough to allow for use on the dermis of a human or animal and repeated exposure to electrical stimuli does not destroy and/or compromise the physical structure of the dosage form. Typically electroresponsive dosage forms including polyacrylic acid (PAA) are too brittle to allow for repeated exposure to electrical stimuli without compromising the physical structure.

The Applicant is not aware of 1-vinylimidazole (1VA) forming part of known hydrogel pharmaceutical dosage forms let alone how 1-vinylimidazole (1VA) would interact with polyethyleneimine (PEI) to form a polymeric hydrogel pharmaceutical dosage form wherein application of the electrical stimulus to the dosage form induces a first conformational change in the dosage form resulting in a release conformation which facilitates an increase in the release rate of the drug from the dosage form to the target site, and wherein cessation of the electrical stimulus to the dosage form induces a second conformational change in the dosage form resulting in a drug containing conformation which facilitates a decrease in the release rate of the drug from the dosage form to the target site. Cessation of the electrical stimulus may also cause the drug release to cease completely.

Drug release from the dosage form to the target site typically takes place via diffusion. The release conformation allows for the drug to be more readily transported out of the dosage form to the target site.

According to a second aspect of this invention there is provided a polymeric hydrogel pharmaceutical dosage form for drug delivery to a target site of a human or animal, the dosage form comprising polyethyleneimine (PEI) and 1-vinylimidazole (1VA) forming an electro responsive matrix. Further the dosage form comprises polyacrylic acid (PAA) and/or polyvinyl alcohol (PVA) at least partially crosslinked with the matrix therein penetrating the matrix to form an interpenetrating polymer network. In use, application of an electrical stimulus to the dosage form induces a first conformational change in the interpenetrating polymer network into a release conformation which facilitates an increase in the release rate of the drug from the dosage form to the target site. Further in use, cessation of the electrical stimulus to the dosage form induces a second conformational change in the interpenetrating polymer network into a drug containing conformation which facilitates a decrease in the release rate of the drug from the dosage form to the target site.

In an example embodiment of the second aspect, the dosage form further comprises a crosslinking agent, preferably the crosslinking agent is N,N′-methylenebisacrylamide. The crosslinking agent may in use crosslink at least one or more of the following group: polyacrylamide (PAA), polyethyleneimine (PEI) and polyvinyl alcohol (PVA). In the manufactured examples described below, the N,N′-methylenebisacrylamide facilitated vinyl addition polymerization.

In a preferred embodiment of the invention the dosage form further comprises a crosslinking initiator, preferably in the form of potassium persulfate.

Typically, the interpenetrating network provides for a high density hydrogel displaying stronger mechanical properties and more efficient drug loading capacity when compared to hydrogels that lack interpenetrating networks.

The dosage form may be a placebo and lack a drug compound, alternatively, the dosage form may be drug loaded and contain a drug compound. Generally, the dosage form is drug loaded. Although it is envisioned that the dosage form could be used to treat a range of medical conditions and/or diseases, typically the dosage form is for use in relieving or ameliorating chronic pain, and the drug may be an analgesic, and is preferably a non-steroidal anti-inflammatory drug (NSAID) such as but not limited to indomethacin. The drug may for example also be morphine, celecoxib and/or fentanyl chloride.

The drug containing conformation slows the release of the drug relative to when the electrical stimulus is applied and may slow drug release to the point where no drug is released whatsoever. Typically, the electrical stimulus increases the rate of diffusion of the drug to the target site. Generally speaking, and as described, illustrated and/or exemplified hereunder in more detail the electrical stimulus is an electric current. The electric current may be applied to the dosage form from about 0.1 seconds to about 60 and any point in between. The electric current may have a voltage of from about 0.3 volts to about 5 volts, and any points in between.

Each component of the dosage form has particular physico-chemical and/or physico-mechanical properties.

In use, the polyethyleneimine (PEI) is electro-conductive allowing for conduction of the electrical stimulus therethrough. Further in use, the polyethyleneimine is electro-responsive such that application of an electrical stimulus induces a structural change in the polyethyleneimine (PEI).

In use, the 1-vinylimidazole (1VA) is electro-conductive allowing for conduction of the electrical stimulus therethrough. Further in use, the 1-vinylimidazole is electro-responsive such that application of an electrical stimulus induces a structural change in the 1-vinylimidazole (1VA). Still further in use, the 1-vinylimidazole (1VA) is a plasticizer so as to increase the plasticity and/or fluidity of the dosage form in use.

In use, the polyvinyl alcohol (PVA) provides mechanical strength and/or robustness.

In use, the polyacrylic acid (PAA) is electro-conductive allowing for conduction of the electrical stimulus therethrough.

The Applicant has noticed that known hydrogels including individually either polyvinyl alcohol (PVA) or polyacrylic acid (PAA) result in hydrogels that show poor viscosity and undesirably high brittleness respectively. Consequently, the hydrogel pharmaceutical dosage form according to the second aspect of the invention, which shows desirable mechanical strength and/or robustness and desirable viscosity in use, was wholly unexpected and surprising.

The Applicant is not aware of 1-vinylimidazole (1VA) forming part of known hydrogel pharmaceutical dosage forms let alone a hydrogel dosage form including 1-vinylimidazole (1VA) and polyethyleneimine (PEI) forming a matrix, and further including polyacrylic acid (PAA) and/or polyvinyl alcohol (PVA) each of which at least partially crosslinks with the matrix and at least partially penetrates the matrix to form an interpenetrating polymer network polymeric hydrogel pharmaceutical dosage form, wherein application of the electrical stimulus to the dosage form induces the first conformational change in the interpenetrating polymer network resulting in the release conformation which facilitates an increase in the release rate of the drug from the dosage form to the target site, and wherein cessation of the electrical stimulus to the dosage form induces the second conformational change in the interpenetrating polymer network resulting in the drug containing conformation which facilitates a decrease in the release rate of the drug from the dosage form to the target site. Cessation of the electrical stimulus may also cause the drug release to cease completely.

Without being limited to theory, the Applicant believes, in terms of the first and second aspects of the invention, that the first conformational change takes place by the electrical stimulus causing the polymer chains of the dosage form to adopt a certain three-dimensional orientation effected by the direction and strength of the electrical stimulus (usually an electric current). The electrical stimulus causes an increase in static energy of the dosage form due to electron transfer resulting in reduced networking among the polymer chains and adoption of the release conformation which facilitates the increase in drug release from the dosage form to the target site when compared to a situation when the electrical stimulus is not applied. The release conformation may provide for channels to form within the hydrogel dosage form which facilitates the release via diffusive means of the drug from the dosage form to the target site. When the electrical stimulus ceases to be applied to the dosage form there is a decrease in static energy resulting in increased networking among the polymer chains and adoption of the drug containing conformation. The increased networking causing the polymer chains to more effectively entrap and/or embed the drug inside the dosage form and prevents its ready release to the target site.

By employing molecular mechanics simulations and subsequent energy/geometry minimizations, complex inter- and intra-molecular interactions were found to occur between polymeric molecules (PAA and PEI), and between polymeric molecules and the plasticizer (PAA, PEI and 1VA) in presence of water molecules under the influence of electric field. The molecular mechanics simulation was carried out in various consecutive steps to generate the final electrosimulation model as follows:
Step 1: Individual molecules namely PAA, PEI and 1VA were generated in vacuum followed by geometrical stabilization;
Step 2: Molecular complexes such as PEI-PAA₂ (two PAA molecules in complexation with one PEI molecule) and PEI-PAA₂-1VA₄ (PEI-PAA₂ molecule in complexation with four 1VA molecules) were generated in vacuum using parallel disposition and were geometrically optimized;
Step 3: PEI-PAA₂-1VA₄ was geometrically optimized under periodic boundary conditions with water as the solvent phase;
Step 4: The solvated PEI-PAA₂-1VA₄ was subjected to electric field in x, y, and z co-ordinate directions at electric field values of 0.1 a.u., 0.3 a.u., and 0.5 a.u. Geometrical optimization was carried out under identical periodic boundary conditions with water as the solvent phase.

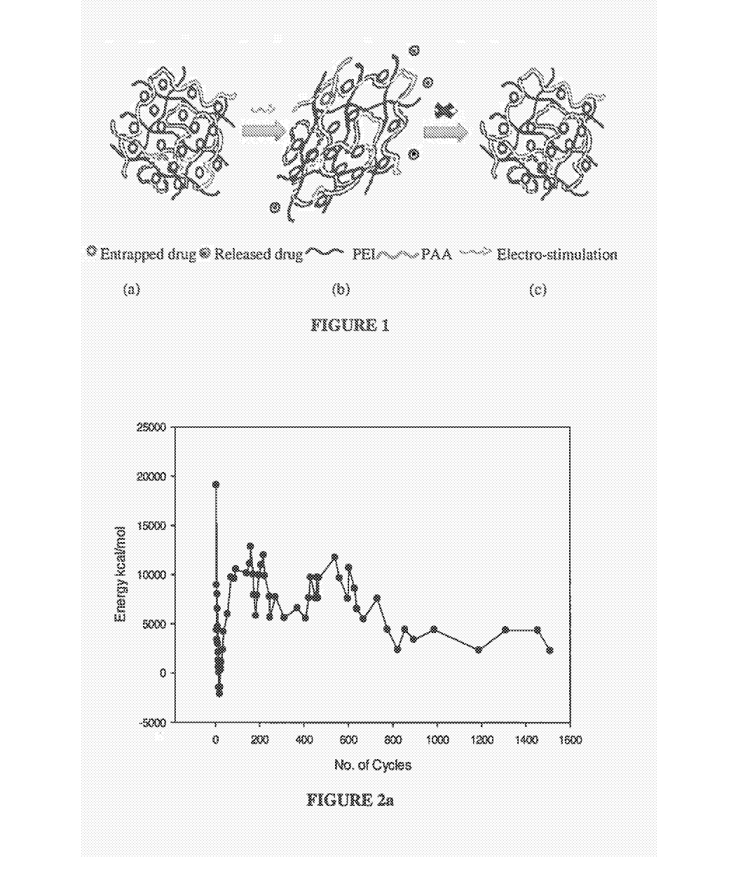

To explain this complex behaviour, a new theory, Pillay's Electro-influenced Geometrical Organization-ReOrganization theory (PEiGOR theory), is presented based on following assumptions and observations as shown in FIG. 1:
1. The Organization—Polymeric chains organise with respect to the direction and strength of electric field: Electric field application→polymer chains organization→increase in static energy due to electron transfer reaction→molecular alignment→planar structural conformation→reduced networking→electroresponsive drug release. This is shown in frame (b) of FIG. 1.
2. The Reorganization—Polymeric chains in assumptions 1 reorganize with respect to surrounding polymer molecules/plasticizer/solvent molecules via “local oriental correlations (LOCs)”: Intrinsic interactions→local oriental correlations→change in reaction co-ordinates→solvent relaxation→polymer chains reorganization→decrease in static energy values→increased networking→drug retention. This is shown in frame (c) of FIG. 1.

Firstly, considering the PEI-PAA₂-1VA₄ molecular build-up in vacuum, the formation of PEI-PAA₂ accompanied with a stabilizing interaction of ≈−30 kcal/mol (Table 1) wherein the van der Waals (vdW) forces played a major role in geometry stabilization with stabilization energy of ≈−30 kcal/mol—meaning that the whole stabilization was brought up by hydrophobic forces in vacuum phase. Interestingly and more convincingly, the formation of PEI-PAA₂-1VA₄ was accompanied with further stabilization of van der Waals component energy reaching to even negative values (=−42 kcal/mol) leading to a contribution of ≈88 kcal/mol towards geometry optimization. In both the cases, the hydrophobic steric interactions (vdW) countered the torsion and stretching caused by the addition of 1-vinylimidazole (1VA) leading to the formation of a well-fitted geometrically-optimized energy-minimized bipolymeric interfacially plasticized structure that acted as the template for further solvated studies under electric field.

TABLE 1
shows inherent energy attributes representing the molecular assemblies modeled
using static lattice atomistic simulations in vacuum and solvated phase.

| Molecular complex | E(V_{Σ})^{a} | E(V_{b})^{b} | E(V_{θ})^{c} | E(V_{φ})^{d} | E(Vᵢⱼ)^{e} | E(V_{hb})^{f} | E(Vₑₗ)^{g} |
|---|---|---|---|---|---|---|---|
| PAA | 76.02 | 8.87 | 43.21 | 10.63 | 13.42 | −0.12 | 0.00 |
| PEI | 28.36 | 1.42 | 5.38 | 9.32 | 12.23 | 0.00 | 0.00 |
| 1VI | 15.68 | 0.05 | 15.05 | ~0.0 | 0.57 | 0.0 | 0.00 |
| PEI-PAA₂ | 150.96 | 19.20 | 92.71 | 31.13 | 8.34 | −0.41 | 0.00 |
| PEI-PAA₂-1VA₄ | 155.81 | 16.58 | 147.04 | 34.16 | −41.96 | 0.00 | 0.00 |
| PEI-PAA₂-1VA₄-H₂O (0.0) | −2645.51 | 37.33 | 170.218 | 40.02 | −67.58 | −0.75 | −2824.74 |
| PEI-PAA₂-1VA₄-H₂O (0.1x) | 2250.91 | 336.831 | 975.802 | 47.44 | 9.30 | −1.21 | −2745.75 |
| PEI-PAA₂-1VA₄-H₂O (0.3x) | 5051.92 | 1198.81 | 2788.45 | 41.58 | 94.02 | −0.29 | −3079.67 |
| PEI-PAA₂-1VA₄-H₂O (0.5x) | 5766.877 | 5321.24 | 8349.21 | 44.18 | 151.34 | −1.51 | −4024.21 |
| PEI-PAA₂-1VA₄-H₂O (0.1y) | 668.41 | 345.73 | 1010.36 | 41.68 | 3.09 | −0.48 | −2837.74 |
| PEI-PAA₂-1VA₄-H₂O (0.3y) | 1853.85 | 1241.53 | 2900.71 | 44.19 | 49.79 | −0.34 | −3279.12 |
| PEI-PAA₂-1VA₄-H₂O (0.5y) | 2956.49 | 5391.06 | 8426.01 | 49.36 | 159.74 | −0.49 | −4263.61 |
| PEI-PAA₂-1VA₄-H₂O (0.1z) | 4141.08 | 348.47 | 1029.64 | 40.59 | 10.06 | −0.59 | −2922.53 |
| PEI-PAA₂-1VA₄-H₂O (0.3z) | 8980.11 | 1232.85 | 2854.01 | 38.91 | 29.98 | −0.41 | −3231.63 |
| PEI-PAA₂-1VA₄-H₂O (0.5z) | 45841.19 | 5409.57 | 8457.95 | 53.28 | 91.98 | −0.44 | −4233.49 |

^{a}total steric energy for an optimized structure
^{b}bond stretching contributions
^{c}bond angle contributions
^{d}torsional contribution arising from deviations from optimum dihedral angles
^{e}van der Waals interactions
^{f}hydrogen-bond energy function
^{g}electrostatic energy

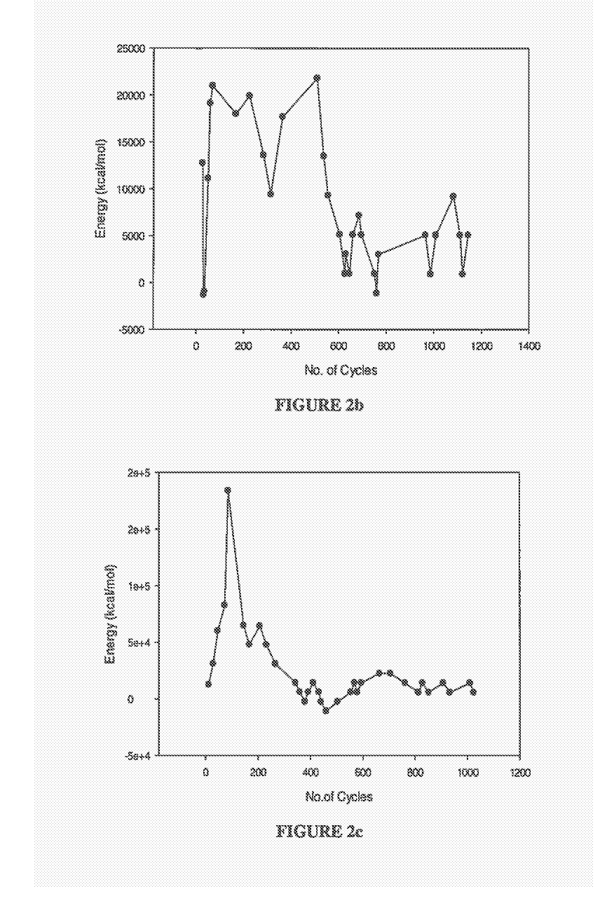

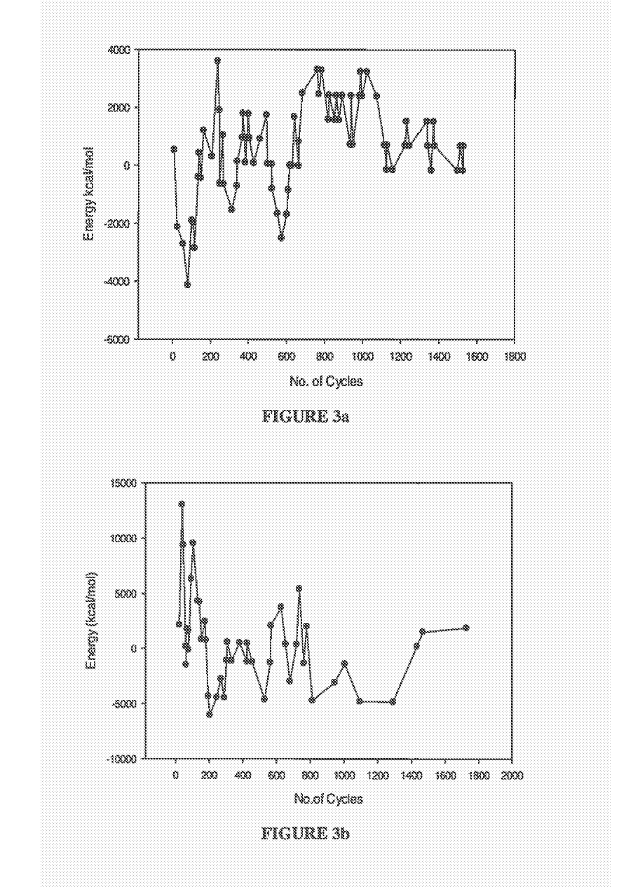

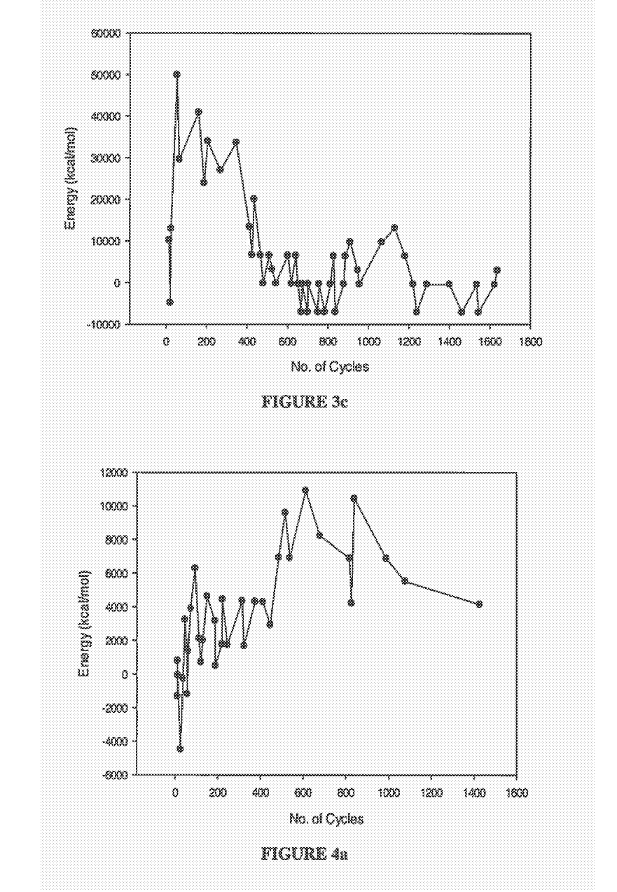

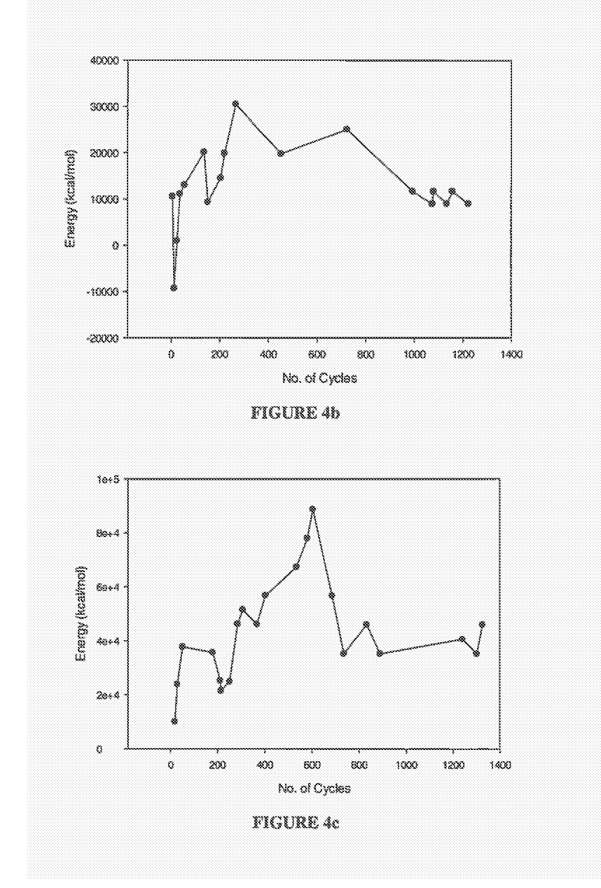

The energy surfaces in FIGS. 2-4 confirm the organization-reorganisation theory where the energy mapping generated for the directional optimization display “fluctuation patterns” representative of the organization-reorganization pattern wherein organization caused a crest in the surface and reorganization resulted in trough formation. Additionally, it is clear from the energy maps shown in FIGS. 2-4 and Table 1 that there is a positive relation between the stabilization energy and the applied electric strength wherein an increase in energy from 2250 kcal/mol to 5766 kcal/mol (x direction); 668 kcal/mol to 2956 kcal/mol (y direction); and 4141 kcal/mol to 45841 kcal/mol (z direction) was observed in case of energy field application at the strengths of 0.1 a.u. to 0.5 a.u., respectively. As the electric potential increased; the stabilization energy also increased which may be due to increased alignment of the electric dipoles with a complete alignment resulting from the forces required to overcome the additional interfaces in the domain structure. The shorter the distance between the point charge from the centre of the molecular complex, the stronger the interactions.

The component energy terms additionally played a deciding role in the molecular simulation and modelling. The component energy values listed in Table 1 represent the average energy values of the fluctuation pattern and have no additive relation to the final optimized value. Considerable hydrogen bonding interactions were observed during the vacuum phase stabilization of the PAA-PEI complex. As expected, the hydrogen bonding (H-bonding) was not constant during the electro simulation as it forms the part of environmental interaction through which the charge transfer occurs. However, it should be noted that the negative H-bonding values were retained throughout the electric direction and field options with values ranging from −1.51 to -0.29 kcal/mol. The electrostatic interaction played a major role in energy stabilization of the final molecular complex with values on higher side of stabilized negative energy scale. Among the destabilization energy terms, all except torsional contribution fluctuated throughout the direction and strength range. From Table 1 it is evident that the spatial Organization might have resulted from the drastic changes in bond stretching and bond angle contributions with small but significant changes in torsional contributions arising from optimum dihedral angles and hydrophobic van der Waals forces with the Reorganization resulting from hydrogen bonding and electrostatic forces as explained above.

The arrangement of plasticizer 1-vinylimidazole (1VA) within polymer sheets resulted in the formation of an electroconductive imidazole ring network across the polymeric architecture of the bipolymeric interfacially plasticized hydrogels. These plasticized microsites were balanced by torsional constraints within the intervening layer which attracted H₂O molecules to hydrate the region, leading to swelling of the hydrogel structure.

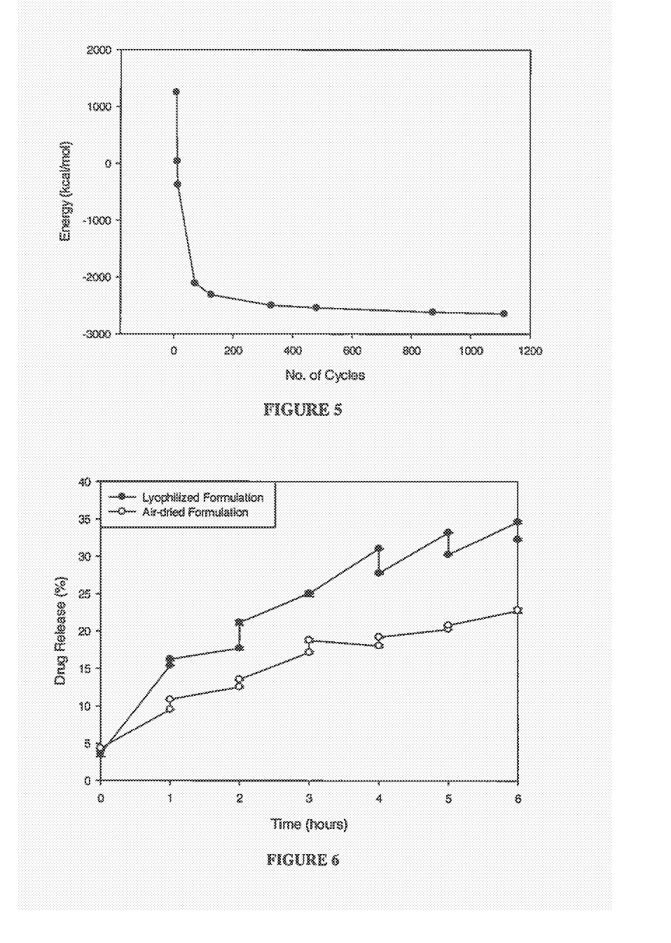

The molecular mechanics simulations under solvated phase displayed some basic similarities of molecular behaviour in all nine cases. 1-vinylimidazole (1VA) molecules appear not to rove around, but instead to tend to drift close to the hydrogen-bonding sites sunken inside the polymer structure. However, the molecules while moving, display a critical “jump diffusional behaviour”—the polymer chains vibrate within a microenvironment for a short period, and then move to new micromolecular sites. These jump-motions are likely to be concentrated along varied locations in the vicinity of electrostatic charged spots attracting the water molecules. However, in contrast, the solvent molecules may exhibit incessant diffusion on the timescale of these simulations. While with no electric field in place; the molecular complex does not show the fluctuation flexibility wherein the molecular components demonstrate a differential spatial variation leading to geometrically optimized and energetically minimized structures via two principle component interactions, one among the polymer/plasticizer molecules and the other among the complex and solvent molecules leading to a well organised and highly stable molecular architecture (FIG. 5).

The dosage form according to the first or second aspect of the invention may form part of a system for transdermal drug delivery, for example, a skin patch assembly. In a preferred embodiment of the invention, the drug application assembly is a microneedle array skin patch assembly. Said skin patch assembly typically forms part of a system for transdermal drug delivery as illustrated in FIG. 11 and described hereunder in more detail.

According to a third aspect of the invention there is provided a method of manufacturing a polymeric hydrogel pharmaceutical dosage form for drug delivery to a target site of a human or animal, the method comprising the following step(s):
(a) mixing together polyethyleneimine (PEI) and 1-vinylimidazole (1VA) to form a first solution;
(b) adding polyvinyl alcohol (PVA) and acrylic acid (AA) to the first solution to form a second solution; and
(c) allowing a polymeric hydrogel to form.

The method according to the third aspect of the invention may comprise an additional step (d), wherein step (d) includes adding a drug to the first solution in order to manufacture a drug loaded polymeric hydrogel pharmaceutical dosage form.

The method may further comprise step (e), wherein step (e) includes adding a crosslinking agent to the second solution, preferably the crosslinking agent may be N,N′-methylenebisacrylamide.

The method may further comprise step (f), wherein step (f) includes adding crosslinking initiator to the second solution, preferably the crosslinking initiator is potassium persulfate.

According to a fourth aspect of the invention there is provided a method of manufacturing a polymeric hydrogel pharmaceutical dosage form for drug delivery to a target site of a human or animal, the method comprising the following step(s):
(a) mixing together polyethyleneimine (PEI), 1-vinylimidazole (1VA) and a drug to form a first solution;
(b) adding polyvinyl alcohol (PVA) and acrylic acid (AA) to the first solution to form a second solution
(c) allowing a polymeric hydrogel to form which contains the drug and is responsive to electrical stimulus.

The method may further comprise step (d), wherein step (d) includes adding a crosslinking agent to the second solution, preferably the crosslinking agent may be N,N′-methylenebisacrylamide.

The method may further comprise step (e), wherein step (e) includes adding crosslinking initiator to the second solution, preferably the crosslinking initiator is potassium persulfate.

According to a fifth aspect of the invention there is provided a method of manufacturing a polymeric hydrogel pharmaceutical dosage form for drug delivery to a target site of a human or animal, the method comprising the following step(s):
(a) preparing a polyvinyl alcohol (PVA) solution to which polyethyleneimine (PEI) and 1-vinylimidazole (1VA) is added to form a first mixture;
(b) adding a drug, acrylic acid and a crosslinking agent to the first mixture; and
(c) allowing a hydrogel to form which contains the drug and is responsive to electrical stimulus.

According to a sixth aspect of the invention there is provided for a method of treating chronic pain in a human or animal, the method comprising the steps of:

applying the polymeric hydrogel pharmaceutical dosage form according to the first and/or second aspect of the invention to a target site of drug delivery; and

applying an electrical stimulus to the dosage wherein application of the electrical stimulus to the dosage form induces a first conformational change in the dosage form resulting in a release conformation which facilitates an increase in the release rate of the drug from the dosage form to the target site, and wherein cessation of the electrical stimulus to the dosage form induces a second conformational change in the dosage form resulting in a drug containing conformation which facilitates a decrease in the release rate of the drug from the dosage form to the target site.

There is provided for the polymeric hydrogel pharmaceutical dosage form, methods to manufacture the same and methods of treating chronic pain as substantially described, illustrated and exemplified herein with reference to any one of the drawings and/or examples.

### EXAMPLES

### 1. Manufacturing and In Vitro Tests

### Materials

Polyethyleneimine (PEI) solution (M_{w} 750,000), 1-vinylimidazole (1VA) (≧99%), Indomethacin (≧99%), polyvinyl alcohol (PVA) (M_{w} 89,000-98,000, 99+% hydrolysed), acrylic acid (AA) (anhydrous, 99%), N,N′-Methylenebisacrylamide (≧99.5%) and potassium persulfate (≧99.0%) were all purchased from Sigma-Aldrich® (St. Louis, USA). All other ingredients were of analytic grade and were used as received.

### Preparation of the Polymeric Hydrogel Pharmaceutical Dosage Form

In order to manufacture the polymeric hydrogel pharmaceutical dosage form according to the invention the following manufacturing method was employed. A 6% polyvinyl alcohol (PVA)-1M sodium hydroxide solution was prepared, to which the polyethyleneimine (PEI) solution and 1-vinylimidazole (1VA) was added to form a mixture. Subsequently, the drug (100 mg-constantly throughout all formulations and for all examples of the drug), was dissolved into the mixture. Acrylic acid was added (0.6 mL). N,N′-Methylenebisacrylamide was then added to facilitate the formation of a interpenetrating hydrogel network (IPHN), instituting vinyl addition polymerization to increase the interconnectivity of the network.

The immediately preceding method produced a drug loaded embodiment of the dosage form. It is envisioned that a placebo embodiment may also be manufactured by omitting the step of adding the drug to the mixture.

Formulations according to the below described Box-Behnken design were formulated for indomethacin. Where other example drugs are utilized they are utilized in the Optimized Formulation but replacing the indomethacin component with another example drug.

### Preparation of 0.01M PBS Solution

In order to simulate the physico-chemical properties of the polymeric hydrogel pharmaceutical dosage form, the hydrogel prepared as described immediately above was exposed to a phosphate buffered saline (PBS) solution, adjusted to physiological pH value (7.4) by the addition the required amount of sodium hydroxide. The preparation of PBS was as described in the British Pharmacopeia (2013). Briefly, 250 mL of 0.2 M potassium dihydrogen phosphate was added to 393.4 mL of 0.1 M sodium hydroxide. Using a pH meter (Eutech pH 510, cyberscan, Singapore), sodium hydroxide was added to the potassium dihydrogen phosphate solution until a final solution of pH 7.4 was made.

### Constraint Optimization of Polymeric Hydrogel Pharmaceutical Dosage Form

In broad terms, the polymeric hydrogel pharmaceutical dosage form according to the invention comprises polyethyleneimine (PEI) and 1-vinylimidazole (1VA).

A model-independent approach (Minitab® V15, Minitab Inc., PA, USA) was used to optimize the dosage form. Statistical optimization using a Box-Behnken design model (Table 2) was therefore employed to ascertain the ideal combination of polymeric species as well as the ideal voltage required capable of attaining desirable drug release, swelling and resilience efficiencies.

TABLE 2
Statistically generated formulations
obtained from a Box-Behnken design.

| For- | | | | | Volt- | 1-Vinylim- | Poly(eth- |
|---|---|---|---|---|---|---|---|
| mula- | Std | Run | Pt | | age | idazole | yleneimine) |
| tion | Order | Order | Type | Blocks | (V) | (mL) | (mL) |
| 1 | 3 | 1 | 2 | 1 | 1 | 1 | 2 |
| 2 | 2 | 2 | 2 | 1 | 5 | 0.1 | 2 |
| 3 | 14 | 3 | 0 | 1 | 3 | 0.55 | 2 |
| 4 | 9 | 4 | 2 | 1 | 3 | 0.1 | 1 |
| 5 | 12 | 5 | 2 | 1 | 3 | 1 | 3 |
| 6 | 8 | 6 | 2 | 1 | 5 | 0.55 | 3 |
| 7 | 4 | 7 | 2 | 1 | 5 | 1 | 2 |
| 8 | 15 | 8 | 0 | 1 | 3 | 0.55 | 2 |
| 9 | 13 | 9 | 0 | 1 | 3 | 0.55 | 2 |
| 10 | 10 | 10 | 2 | 1 | 3 | 1 | 1 |
| 11 | 5 | 11 | 2 | 1 | 1 | 0.55 | 1 |
| 12 | 1 | 12 | 2 | 1 | 1 | 0.1 | 2 |
| 13 | 7 | 13 | 2 | 1 | 1 | 0.55 | 3 |
| 14 | 11 | 14 | 2 | 1 | 3 | 0.1 | 3 |
| 15 | 6 | 15 | 2 | 1 | 5 | 0.55 | 1 |

All of the other hydrogel components and the drug (100 mg) remained constant throughout all the formulations. The only variations were the voltage, polyethyleneimine (PEI) and 1-vinylimidazole (1VA) in each formulation.

Construction of Calibration Curve for the Ultraviolet Spectrophotometric Determination of Indomethacin (Example Drug) Release from the Polymeric Hydrogel Dosage Form According to the Invention

An ultraviolet spectrophotometric scan was run to determine the maximum wavelength for Indomethacin absorption in phosphate buffered saline (PBS). Using Ultraviolet-visible (UV) spectroscopy, it was found that Indomethacin exhibits a maximum wavelength at λ₃₂₀. This is consistent with the literature published on Indomethacin 319 nm absorption peak (Forster et al., 2001; Anoopkumar-Dukie, 2003; Kamal et al., 2008). Using a series of known concentrations of Indomethacin in PBS, a calibration curve at the aforementioned wavelength was constructed. The linear curve was plotted with the observed absorbance of Indomethacin as the dependent variable and the concentration of Indomethacin as the independent variable. A statistical representation of the degree at which the function correlates the set of values (R² was computed for the curve. The curve could be described by the straight line equation y=1.7074x+0.581 (R²=99).

### Determination of the Effect of Aluminium Foil on the Drug Release Profiles of the Polymeric Hydrogel Pharmaceutical Dosage Form Using Indomethacin as the Example Drug

Aluminium foil was used as means of method modulation to determine the effects on drug release. In vitro drug release studies on the polymeric hydrogel pharmaceutical dosage form were performed as detailed:

The polymeric hydrogel pharmaceutical dosage form Formulations, as per Table 2, {each of the Formulations were tested} were immersed in 20 mL of phosphate buffered saline PBS (pH 7.4; 37° C.), and a potential difference of 3V was applied to each corresponding Formulation (as per Table 1) respectively using a potentiostat/galvanostat (PGSTAT302N, Autolab, Utrecht, Netherlands).

An aluminium foil covered each dosage form and onto which two electrodes were directly placed. A 5 platinum electrode served as the cathode and the anode, a 5 mm gold electrode. A potential difference was maintained between the two electrodes. The potential difference was maintained for one minute and 2 mL PBS was sampled at hourly intervals with the replacement of fresh PBS medium after the application of the electrical stimulation in order to maintain sink conditions. The same procedure was performed for up to 6 hours and samples were analysed for indomethacin content using UV/visible spectroscopy. It was determined that the presence of aluminium foil increased the release rate when compared to a control sample.

The lyophilized formulation, although displaying a greater increase in drug release, is no longer electro-responsive from hour 3 (FIG. 6) indicating possible conformational changes of the hydrogel matrix induced by the process of dehydration. The initial display of an increase in release is due to the osmotic effect of the liquid penetrating the hydrogel matrix due to the concentration gradient. The enhanced release seen with the lyophilized formulation is due to the larger diffusion gradient caused by lyophilization and subsequent rehydration in solution. Compared to the lyophilized formulation, the air-dried formulation does, however, display continuous electro-responsive ability, possibly as a result of the osmotic gradient. The Formulation used for lyophilisation was a replicate of Formulation 1 from Table 2.

### In Vitro Drug Release Analysis (Indomethacin)

Dissolution is frequently the rate-controlling step in drug absorption of poorly soluble drugs. In the commonly used dissolution methods the concentration of dissolved substance is measured in the bulk release media. In principle, faster and more detailed studies of drug dissolution may be achieved if the dissolution can be measured at the solid-liquid interface. With UV imaging it is possible to measure the intensity of light passing through an area of a quartz tube as a function of position and time. Thus, UV imaging facilitates quantification of drug substances in solution immediately adjacent to the solid material and recording of concentration gradients. In vitro drug release studies on the polymeric hydrogel pharmaceutical dosage forms were performed as detailed hereunder:

The Formulations, as per Table 2, having indomethacin as the example drug, were immersed in 20 mL of phosphate buffered saline PBS (pH 7.4; 37° C.), and varying potential differences (as per the Box-Behnken design of Table 2) was applied to each corresponding formulation respectively using a potentiostat/galvanostat (PGSTAT302N, Autolab, Utrecht, Netherlands).

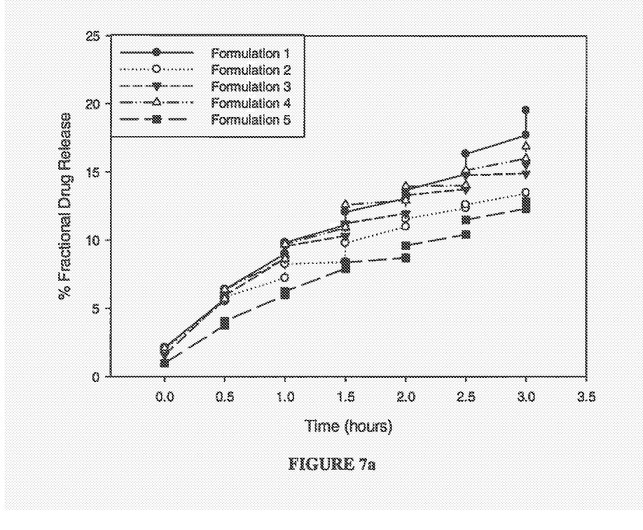

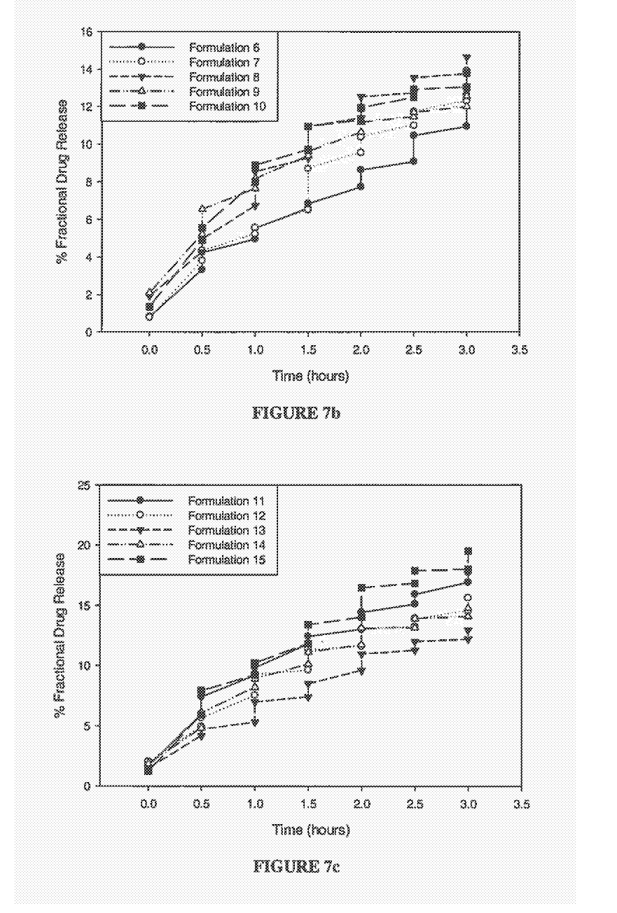

An aluminium foil covered each Formulation on which two electrodes were directly placed. A 5 mm platinum electrode served as the cathode and a 5 mm gold electrode served as the anode. A potential difference was maintained between the two electrodes. The potential difference was maintained for one minute and 2 mL PBS was sampled at hourly intervals with the replacement of fresh PBS medium after the application of the electrical stimulation in order to maintain sink conditions. The same procedure was performed for up to 3 hours and samples were analysed for indomethacin content using UV/visible spectroscopy (IMPLEN Nanophotmeter™, Implen GmbH, Munchen Germany). The resultant drug release profiles obtained are shown in FIG. 7 a-c. Analysis was conducted in triplicate. The average drug release values obtained in each design formulation per electro-stimulus spike was recorded as per Table 3.

TABLE 3
Average drug release values obtained after electro-stimulation as per
Box-Behnken design of Table 2 (Indomethacin as the example drug)

| Formulation | Average Drug Release (mg) |
|---|---|
| 1 | 1.657586 |
| 2 | 0.906829 |
| 3 | 1.041786 |
| 4 | 1.353905 |
| 5 | 0.726095 |
| 6 | 0.986686 |
| 7 | 1.07409 |
| 8 | 1.200152 |
| 9 | 1.094367 |
| 10 | 1.199324 |
| 11 | 1.210552 |
| 12 | 1.233895 |
| 13 | 1.237705 |
| 14 | 1.370233 |
| 15 | 1.725062 |

Drug release from each pharmaceutical hydrogel dosage form will generally effected by hydrogel swelling, diffusion, degradation of labile covalent bonds or reversible drug-polymer interactions with the device geometry significantly influencing the resulting drug release kinetics as well (Zarzycki et al., 2010).

### Swelling Studies Polymeric Hydrogel Pharmaceutical Dosage Forms

Peppas (2000) and co-workers purport that the swelling of hydrogels is pre-determined by the crosslinking ratio. By determining the degree of hydration and/or swelling allows for an understanding of the transport of small drug molecules through the hydrogel matrix. Hydration strongly correlates with in vitro and in vivo biocompatibility as it influences the elastic modulus and surface properties such as wettability (Guiseppi-Elie, 2010). Water may penetrate a gel network causing swelling and thus giving the hydrogel its form. Thus, swelling studies form the basis for establishing a gels nature (Samui et al., 2007; Moya-Ortega et al., 2010; Shalvari et al., 2010). The absolute change in volume is by no means insignificant-dimensional changes of say some percents are quite usual (Bajpai et al., 2008). Swelling attributes of a hydrogel are a key parameter because the equilibrium swelling ratio influences many properties of the hydrogel such as controlled drug release mechanisms as well as determining its potential applications (Peng et al., 2009; Frutos et al., 2010; Ferrero et al., 2010). The hydrogel samples were analysed using the Karl Fischer (Mettler Toledo V30 Volumetric KF Titrator, Mettler Toledo Instruments Inc., Greifensee, Switzerland) as well as the conventional approach using the weight of the hydrogel, where: The gel sample was weighed before submersion into PBS and then again after 24 hours. The gel was taken out and surface water removed followed by the determination of equilibrium swelling ratio. The equilibrium swelling ratio (ESR) was calculated using equation 1:

ESR=(W₁−W₀)/W₀ Equation 1

Where W₀ is the weight of the dried hydrogel and W₁ is the weight of the superabsorbent hydrogel. The conventional method was also used to analyze the degree of swelling in comparison with the Karl Fischer titrator. In addition to determining the degree of swelling, the two methods were compared as well (Table 4).

TABLE 4
Comparison of Karl Fischer Titrator and conventional
swelling determination methods

| | KF Titrator | Conventional Swelling |
|---|---|---|
| Formulation | Swelling (%) | Method (%) |
| 1 | 27.2870 | 28.9043 |
| 2 | 28.0050 | 33.8035 |
| 3 | 49.7987 | 47.8983 |
| 4 | 30.7833 | 26.7347 |
| 5 | 22.2990 | 23.5085 |
| 6 | 47.9100 | 46.1225 |
| 7 | 45.8576 | 48.9300 |
| 8 | 30.8643 | 31.0880 |
| 9 | 47.6897 | 45.5363 |
| 10 | 31.7073 | 28.8998 |
| 11 | 39.2387 | 41.1979 |
| 12 | 31.882 | 33.9135 |
| 13 | 31.2365 | 35.9877 |
| 14 | 48.2246 | 50.5464 |
| 15 | 40.5819 | 42.6718 |

As can be seen by the results, the two methods are similar. The KF method does however, provide a more accurate result as the conventional method is subject to variability in terms of weighing the sample on the scale and removing excess fluid (Belma, 2000).

{All formulations were drug loaded with indomethacin unless otherwise specified}

### Textural Profile Analysis to Determine the Physico-Mechanical Behaviour of the Polymeric Hydrogel Pharmaceutical Dosage Form

A Texture Analyzer (TA.XTplus Stable Microsystems, Surrey, UK) was used to characterize the Formulations of Table 2 in terms of matrix resilience. Computations of matrix resilience for the samples were performed using Force-Time profiles (N=3). Table 5 outlines the TA settings utilized in the calculation of the matrix resilience values of the formulations of the experimental design.

TABLE 5
Parameters employed in the measurement of hydrogel dosage
form samples employing the texture analyzer.

| Parameter | Settings |
|---|---|
| Test Mode | Compression |
| Pre-Test Speed | 1.0 mm/sec |
| Test Speed | 1.5 mm/sec |
| Post-Speed Speed | 1.5 mm/sec |
| Target Mode | Strain |
| Strain | 10% |
| Trigger Type | Force |
| Trigger Force | 0.05N |
| Probe type | 10 mm Delrin cylinder probe |

A typical force-time profile generated for computation of each Formulation's matrix resilience was generated. The obtained resilience values are summarized in Table 6.

TABLE 6
Calculated matrix resilience values (N = 3) for the
Formulations 1-15 of Table 2.

| Formulation | Calculated Matrix Resilience (%) |
|---|---|
| 1 | 53.85 |
| 2 | 100 |
| 3 | 100 |
| 4 | 83.33 |
| 5 | 100 |
| 6 | 97.22 |
| 7 | 38.81 |
| 8 | 100 |
| 9 | 100 |
| 10 | 100 |
| 11 | 100 |
| 12 | 100 |
| 13 | 100 |
| 14 | 100 |
| 15 | 22.16 |

{All formulations were drug loaded with indomethacin unless otherwise specified}

### Optimization of Formulation Responses

A single, optimal formulation was developed subsequent to constraint optimization of desirable drug release, swelling and matrix resilience efficiencies. The response optimization was carried out utilizing statistical software (Minitab®, V14, Minitab Inc®, PA, USA) to determine the optimum chemical composition and also the optimum voltage required to attain the desired drug release.

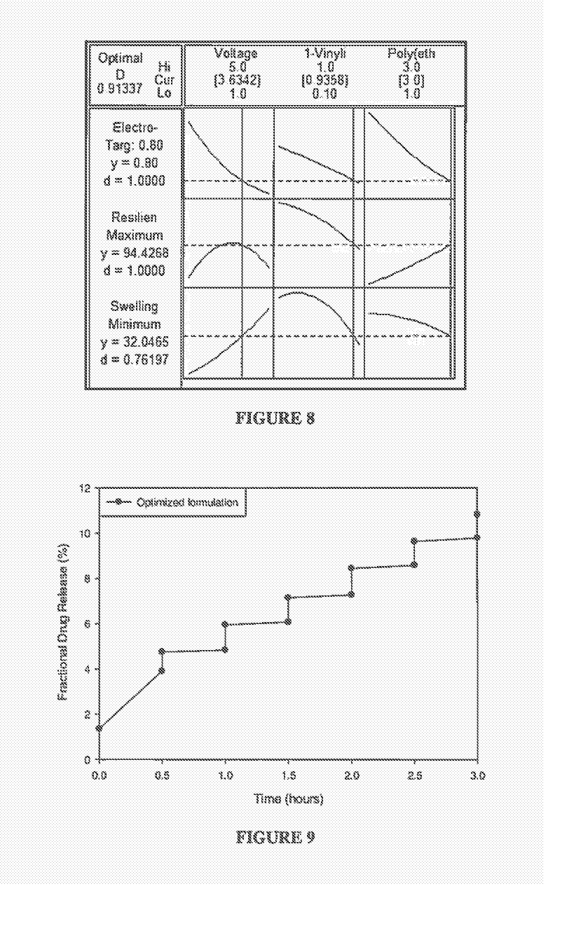

FIG. 8 depicts the desirability plots of each constraint for the single optimal formulation. Constraint settings utilized are shown in the following table. The optimal levels of the independent variables that would achieve the desired drug release, swelling and matrix resilience characteristics are depicted in Table 7. The Optimized Formulation comprised 20 mL of a 6% polyvinyl alcohol (PVA)-1M sodium hydroxide solution (1.2 g polyvinyl alcohol (PVA) dissolved in a sodium hydroxide solution comprising of 40 g sodium hydroxide in 1 L deionized water, polyethyleneimine (PEI) solution (3 mL), 1-vinylimidazole (1VA) solution (0.9358 mL), indomethacin (100 mg), Acrylic acid (0.6 mL), N,N′-Methylenebisacrylamide (100 mg), and a potassium persulfate (KPS) solution of 50 mg in 1 mL water. An applied voltage of 3.63 V was used to attain the drug release of ±0.8% per electro-stimulation.

TABLE 7
Formulation constraints utilized for response optimization.

| Responses | Limits |
|---|---|
| Drug Release | Maximize |
| Swelling | Minimize |
| Matrix Resilience | Maximize |

Construction of Calibration Curve for the Ultraviolet Spectrophotometric Determination of Morphine Hydrochloride Release from the Polymeric Hydrogel Dosage Form According to the Invention

An ultraviolet spectrophotometric scan was run to determine the maximum wavelength for Morphine HCL absorption in phosphate buffered saline (PBS). Using Ultraviolet-visible (UV) spectroscopy, it was found that Morphine HCL exhibits a maximum wavelength at λ₂₇₈. This is consistent with the literature published on Morphine HCL 285 nm absorption peak (Morales et al., 2004; Morales et al., 2011).

Using a series of known concentrations of Morphine HCL in PBS, the calibration curve at the fore mentioned wavelength was constructed. The linear curve was plotted with the observed absorbance of Morphine HCL as the dependent variable and the concentration of Morphine HCL the independent variable. A statistical representation of the degree at which the function correlates the set of values (R2 was computed for the curve. The curve could be described by the straight line equation y=3.020x+0.068 (R²=0.99).

Construction of Calibration Curve for the Ultraviolet Spectrophotometric Determination of Celecoxib Release from the Polymeric Hydrogel Dosage Form According to the Invention

An ultraviolet spectrophotometric scan was run to determine the maximum wavelength for Celecoxib absorption in phosphate buffered saline (PBS). Using Ultraviolet-visible (UV) spectroscopy, it was found that Celecoxib exhibits a maximum wavelength at λ₂₀₈. This is consistent with the literature published on Celecoxib 215 nm absorption peak (Frank et al., 2004). Using a series of known concentrations of Celecoxib in PBS, the calibration curve at the aforementioned wavelength was constructed. The linear curve was plotted with the observed absorbance of Celecoxib as the dependent variable and the concentration of Celecoxib the independent variable. A statistical representation of the degree at which the function correlates the set of values (R² value) was computed for the curve. The curve could be described by the straight line equation y=1.678+0.0493 (R²=0.99).

Construction of Calibration Curve for the Ultraviolet Spectrophotometric Determination of Fentanyl Citrate Release from the Polymeric Hydrogel Dosage Form According to the Invention

An ultraviolet spectrophotometric scan was run to determine the maximum wavelength for fentanyl citrate absorption in phosphate buffered saline (PBS). Using Ultraviolet-visible (UV) spectroscopy, it was found that fentanyl citrate exhibits a maximum wavelength at λ₂₀₃. This is consistent with the literature published on fentanyl citrate 258 nm absorption peak (Almousa et al., 2011). Using a known series of concentrations of fentanyl citrate in PBS, the calibration curve at the aforementioned wavelength was constructed. The linear curve was plotted with the observed absorbance of fentanyl citrate as the dependent variable and the concentration of fentanyl citrate the independent variable. A statistical representation of the degree at which the function correlates the set of values (R² value) was computed for the curve. The curve could be described by the straight line equation y=0.0984x+0.0044 (R²=0.99).

### In Vitro Drug Release Analysis of Optimized Formulation

In vitro drug release studies on the polymeric hydrogel pharmaceutical dosage forms were performed as detailed hereunder. The same Optimized Formulation comprising 20 mL of a 6% polyvinyl alcohol (PVA)-1M sodium hydroxide solution (1.2 g polyvinyl alcohol (PVA) dissolved in a sodium hydroxide solution comprising of 40 g sodium hydroxide in 1 L deionized water, polyethyleneimine (PEI) solution (3 mL), 1-vinylimidazole (1VA) solution (0.9358 mL), indomethacin (100 mg), Acrylic acid (0.6 mL), N,N′-Methylenebisacrylamide (100 mg), and a potassium persulfate (KPS) solution of 50 mg in 1 mL water. An applied voltage of 3.63 V was used to attain the drug release of ±0.8% per electro-stimulation and was tested three times:

The Optimized Formulations 1, 2 and 3 were immersed in 20 mL of phosphate buffered saline PBS (pH 7.4; 37° C.), and varying potential differences (as per Box-Behnken design in Table 2) was applied to each corresponding formulation respectively using a potentiostat/galvanostat (PGSTAT302N, Autolab, Utrecht, Netherlands). An aluminium foil covered each Optimized Formulation 1, 2 and 3 on which two electrodes were directly placed. A 5 mm platinum electrode served as the cathode and a 5 mm gold electrode served as the anode. A potential difference was maintained between the two electrodes. The potential difference was maintained for one minute and 2 mL PBS was sampled at hourly intervals with the replacement of fresh PBS medium after the application of the electrical stimulation in order to maintain sink conditions. The same procedure was performed for up to 3 hours and samples were analysed for indomethacin content using UV/visible spectroscopy (IMPLEN Nanophotmeter™, Implen GmbH, Munchen Germany). The resultant drug release profiles obtained are shown in FIG. 9. Analysis was conducted in triplicate.

### In Vitro Drug Release Studies on the Polymeric Hydrogel Dosage Form According to the Invention

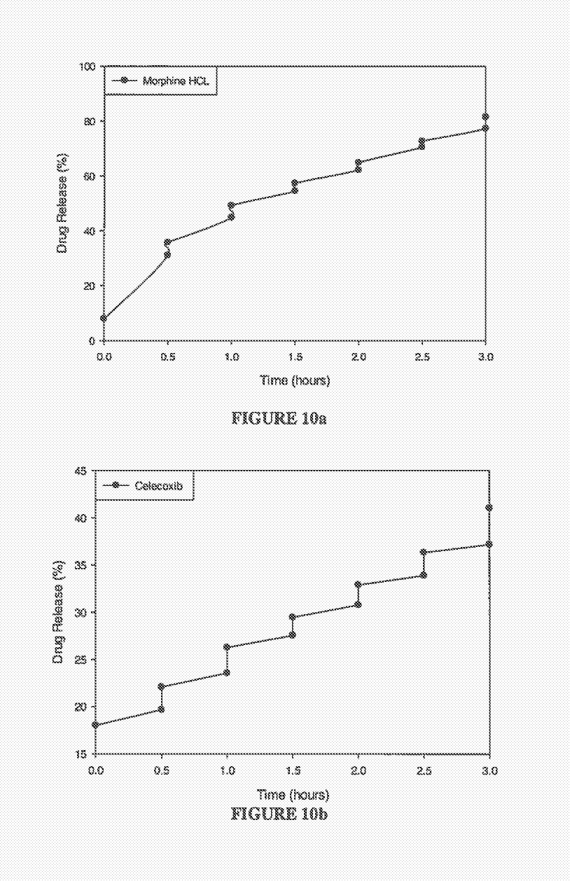

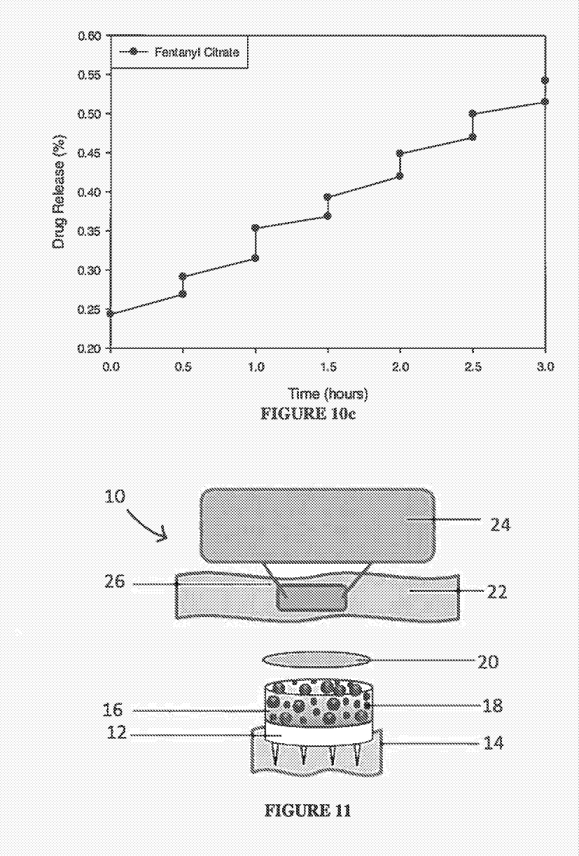

Further in vitro studies were carried out on the optimized formulation containing independently morphine HCL, celecoxib and fentanyl citrate in order to determine the versatility of the formulation. The drug release studies were carried out as previously mentioned and illustrated in FIG. 10 a-c.

### Conclusions

The polymeric hydrogel dosage form according to the invention has successfully been used to deliver the example drugs in an electro-responsive manner.

### 2. Animal Studies

Animal studies were conducted to test the polymeric hydrogel pharmaceutical dosage form according to the invention. An example embodiment of how the dosage form was applied to the skin of an animal is shown in FIG. 11 as part of a system for transdermal drug delivery 10. FIG. 11 shows a microneedle array 12 adjacent to exposed skin 14 of an animal. The microneedle array 12 in use pierces the exposed skin creating channels in the skin which facilitates the transdermal delivery of an example drug compound into the systemic circulation of the animal. Superposed on top of the microneedle array 12 is the polymeric hydrogel pharmaceutical dosage form 16 having therein the example drug compound 18. A piece of aluminium foil 20 is placed on top of the dosage form 16. The microneedle array 12, dosage form 16 and foil 20 are secured against the skin 14 using a plaster 22. An electro-stimulating device 24 is connected to be in electrical communication with the foil 20 via an electrode 26.

It is to be appreciated that the system 10 is merely an example embodiment of how to apply the polymeric hydrogel pharmaceutical dosage form according to the invention. It is to be understood that a person skilled in the art could readily conceive of alternative embodiments of such a system for transdermal drug delivery. Further, the polymeric hydrogel pharmaceutical dosage form according to the invention is not limited to use in transdermal applications, although it is shown hereunder that said dosage form is indeed useful in such application.

### Design of the In Vivo Experimental Study

A total of 18 Sprague-Dawley rats with an initial weight of ˜225 g were used in the study. The rats were randomly assigned to 3 groups (n=6). The experimental procedures for each of the groups were run as described hereunder and as illustrated in FIG. 6:

### Group 1: Conventional Study

The rats in this group received IV administration of indomethacin (0.8 mg/100 g body weight) 15 min prior to blood sampling (Lacroix and Rivest, 1996).

### Group 2: Experimental Study

The rats in this group received the drug-loaded polymeric hydrogel pharmaceutical dosage form according to the invention (Indomethacin as the example drug) device in between the shoulder area. The device was subjected to electro-stimulation of 3.63V at the required time intervals. The intensity used falls within range of voltages that are acceptable to be used in a rat model (Mayer and Westbrook, 1983).

### Group 3: Placebo Study

The rats were assessed for any signs of discomfort or behavioural changes and received the system applied to their dermis but without electro-stimulation.

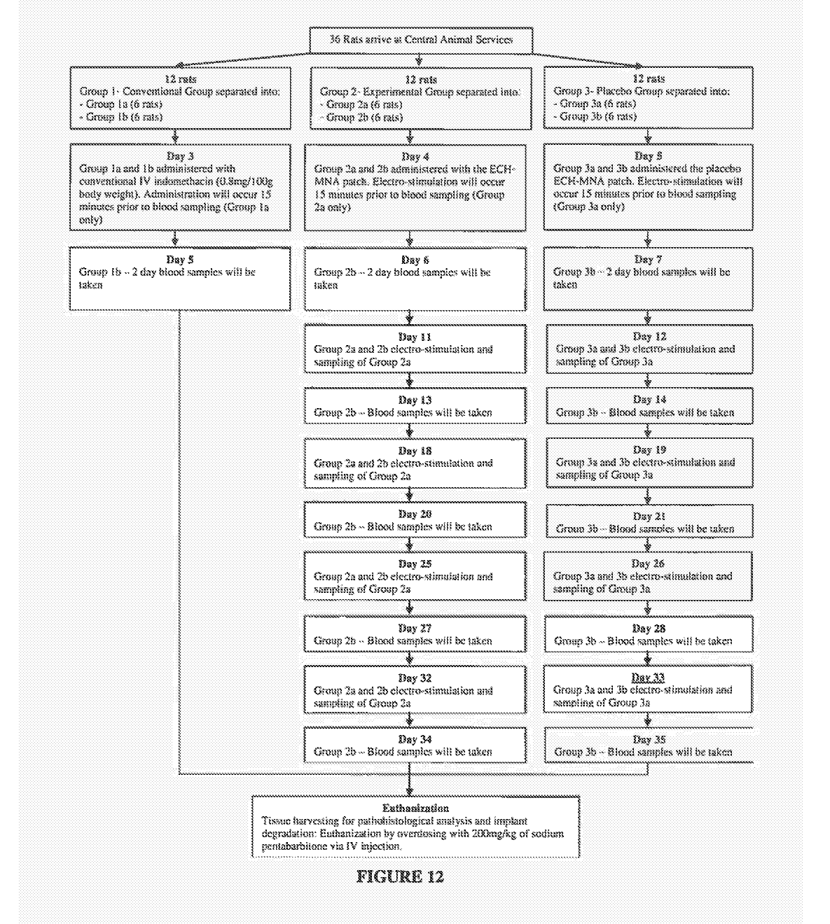

Each group contained two subgroups (a) and (b), each subgroup having three (3) rats. All 3 rats in each subgroup were administered with the respective delivery system at Day 3, 4 and 5 for Groups 1, 2 and 3 In Group 1, the blood sampling time point for 3 rats (Group 1a) is at day 3 prior to and after administration with blood samples taken 2 days later in the remaining 3 rats (Group 1b). In Group 2, blood sampling occurred at weekly intervals with the first dose given at Day 4 and the first blood samples taken prior to and 15 minutes after electro-stimulation. Electro-stimulation and blood samples was subsequently taken for these 3 rats at Days 11, 18, 25 and 32 prior to and after electro-stimulation. The remaining 3 rats in the group (Group 2b) that were administered with the delivery system at Day 4 were electro-stimulated at Days 11, 18, 25 and 32; however blood samples were taken 2 days after electro-stimulation on Days 6, 13, 20, 27 and 34. Sampling at these time points were taken to prove the presence of indomethacin in the rat's cardio-vascular system after 2 days (t_{1/2}˜7-10 hr) and will not be present at the next weekly electro-stimulation (Elahi et al., 2009). Furthermore, the reason for staggering the sampling points as well as using the 3 rats in Group 2b is due to the inability of rats to provide more than 1 mL of blood per week excluding use of the rats in Group 2a. The total number of blood samples, per rat was limited to 10 samples during the period of the study. This procedure will be repeated for Group 3. The timeline depicting the electro-stimulation as well as the blood sampling points for each group can be found in FIG. 12.

Prior to the application of the system, the dorsal surface of the rats were shaved whilst they were under anaesthetic so as to prevent any undue distress. It should be noted that the absence of a hair coat mimics the human skin better than hairy skin as evident by the numerous studies using hairless species, such as nude mice and hairless rats (Simon and Maibach, 1998). The system was placed onto the area between the shoulder blades and was secured through the use of a plaster. The rat was bandaged around the torso in order to prevent removal of the device as a result of scratching. The hydrogel dosage form according to the invention was hydrated using double de-ionized water and aluminium foil, serving as the conducting interface, was placed onto the microneedle array prior to electro-stimulation, as shown in FIG. 11.

### Procedure for Blood Collection, Sampling and Treatment

A plastic restraint device was used to allow for easy blood collection, allowing minimal movement and thus preventing any undue pain through self-inflicted injury. Animal restraint time was reduced to an absolute minimum on welfare grounds. The blood collection technique employed use of the tail vein (Hoff, 2000 & Lawson, 2000). Prior to blood collection, the tail was warmed by dipping it into slightly heated water to induce vessel dilation and subsequently, easy blood collection. Blood samples (0.5 mL) were collected using a 1 mL syringe pre-flushed with heparin.

After withdrawal, blood samples were placed into 2 mL polypropylene tubes that were also pre-flushed with heparin. Blank blood for base-line data was withdrawn 1 week prior to application of the device.

After collection, the blood samples were centrifuged at 12000 RCF (TG16-WS, Nison Instrument Limited, Shanghai, China) for 10 min. The supernatant, containing the plasma, was carefully aspirated and transferred into a clean collection tube and frozen at −80° C. immediately until further analysis. The conventional group received 0.4 mL indomethacin through the tail vein. At 15 minutes and 48 hrs, blood was withdrawn and treated as described.

### Quantification of the In Vivo Release of the Anti-Inflammatory Agent Using Ultra-Performance Liquid Chromatography Analysis

An ultraperformance liquid chromatographic (UPLC) method was developed employing a Waters® ACQUITY™ LC system (Waters®, Milford, Mass., USA) coupled with a photodiode array detector (PDA), and Empower® Pro Software (Waters®, Milford, Mass., USA). The UPLC was fitted with an Aquity UPLC® High Strength Silica (HSS) RP18 column, with a particle size of 1.8 μm and pore size of 100 Å. An isocratic method with a run time of 7 min was developed using acetonitrile and 0.1% v/v formic acid in double deionized water as the mobile phase in a 50:50 ratio. The flow rate was 0.1 mL/min with an injection volume of 10 μL. The PDA detector was set at 254 nm. Naproxen sodium was used as the internal standard (IS). The assay procedure was performed at room temperature (21±0.5° C.).

### Sample Preparation of Plasma Samples Utilizing Liquid-Liquid Extraction

Indomethacin is highly protein bound (Raveendran et al., 1992) thus a liquid-liquid plasma extraction procedure was applied to the rat plasma containing indomethacin. The simple technique is both rapid and relatively cost effective per sample as compared to other techniques and near quantitative recoveries (90%) of most drugs can be obtained (Prabu and Suriyaprakash, 2012). Stored and frozen study samples were allowed to environmentally equilibrate at room temperature (25±0.5° C.). Aliquots of plasma (500 μL) were transferred into polypropylene tubes. Acetonitrile (500 μL) was added to the tubes and the plasma solution vortexed for 2 min for precipitation of the plasma proteins. Acetonitrile (500 μL) was subsequently added to the samples and vortexed again for 2 min. The mixture was then centrifuged at 12000RCF (Nison Instrument Limited, Shanghai, China) for 10 min. The supernatant was subsequently removed and filtered through 0.22 μm Cameo Acetate membrane filters. To an aliquot of 10 μL plasma, the internal standard solution (10 μg) was added and vortexed for 2 min. The final solution was transferred into Waters® certified UPLC vials for analysis. Measurements were conducted on each three samples in triplicate.

### Pharmacokinetic Analysis for the Establishment of an In Vitro-In Vivo Correlation

WinNonLin® software (V5.2.1 with IVIVC Toolkit Build 2008033011, Pharsight Software, Statistical Consultants Inc., Apex, NC, USA) was used as a tool for pharmacokinetic computations and estimation of all the pertinent pharmacokinetic parameters for the development of a Level A time-scaled in vitro-in vivo correlation. Input data comprised in vitro indomethacin release data obtained from the device as well as pharmacokinetic data obtained from the described vivo experimental protocol of the transdermal system applied transdermally to six Sprague Dawley rats whereby blood plasma samples were obtained and analyzed via UPLC over a period of 35 days.

### Results and Discussion

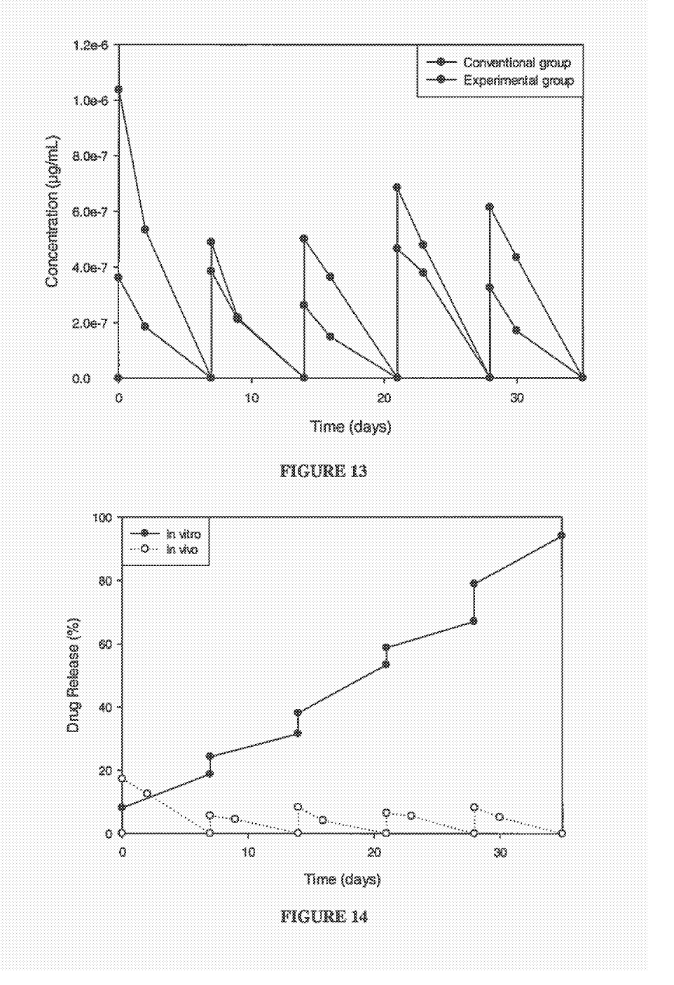

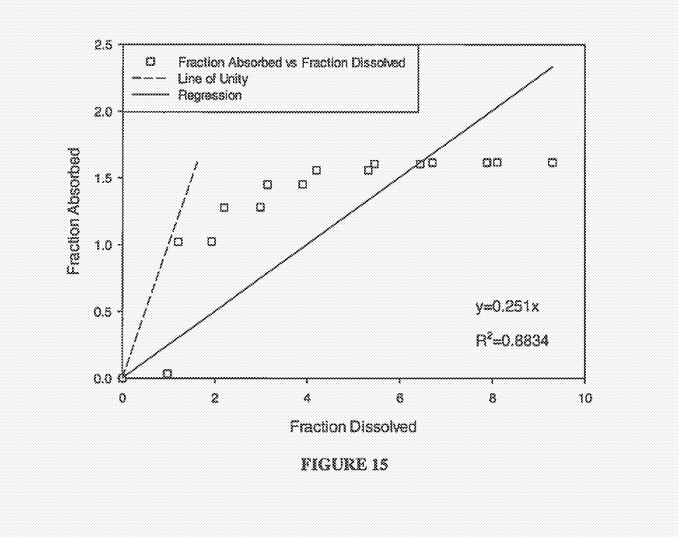

The in vivo release profiles of indomethacin from the transdermal system as well as from the intravenously administered conventional are depicted in FIG. 13. The profiles display contrasting results where the transdermal system displayed significantly higher levels of release in the plasma as compared to the conventional delivery system. Peak levels of 1.0373×10⁻⁶ μg/mL of indomethacin were reached after electro-stimulation. Furthermore, drug was released in desired electro-responsive manner with the release profiles depicting no irregularities or fluctuations. Although lower levels of indomethacin were obtained, the rat does however have a higher metabolism and lower blood volume compared to that in humans. No visible signs of discomfort or abnormal behavior were observed in the study suggesting that the doses entering the systemic circulation were not significant enough to cause any side-effects and thus reiterate the success of the drug delivery system.

### Establishment of an In Vitro-In Vivo Correlation

The IVIVC regarding a transdermal drug delivery system of this nature has not been examined apparent by the lack of available literature. An extravascular single-dose, first-order absorption one compartment model without lag was selected for indomethacin for development of the IVIVC model, being the best fit as predicted by initial pharmacokinetic analysis. A Level A correlation was developed by calculating the amount of indomethacin absorbed using the Wagner Nelson method using the linear trapezoidal rule. To ascertain that a level A IVIVC was obtained, the percentage of drug absorbed up to time t was plotted versus the amount of drug released in vitro (FIG. 14).

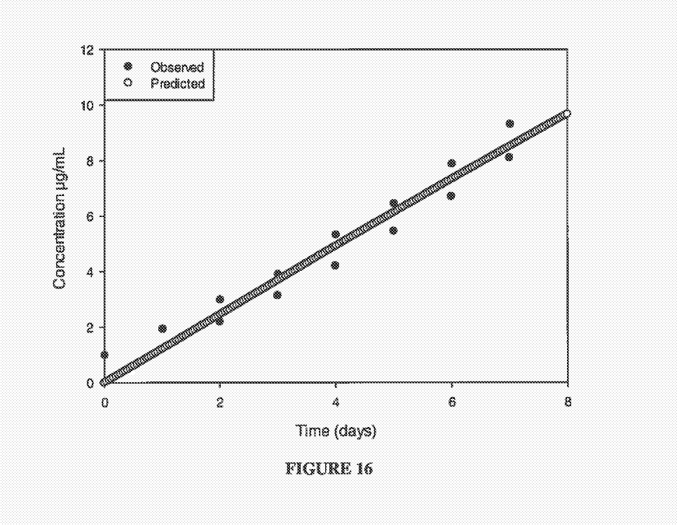

The initial release of ±10% observed after electro-stimulation has allowed for the drug to be maintained within the rat's therapeutic levels. Level A analysis yielded an R² value of 0.8834 indicating that the in vitro data was predictive of in vivo data with 88.34% accuracy. No suitable predictions could be established due to the pulsatile nature of the electro-responsive system (FIG. 16) This does not in any way negatively effect the interpretation of the usefulness of the transdermal system. To attain accuracy of the in vivo-in vitro correlation the Applicant made use of an existing kinetic model as no model exists to explain a transdermal system of this nature. However, the imperfect superimposability observed in the in vitro/in vivo plot may result from residual release from the polymeric hydrogel pharmaceutical dosage form according to the invention, accounting for the increase in drug release after electro-stimulation on day 0 to day 7. The initial in vivo release of indomethacin from the transdermal system can be accounted for by the size of the rats as they generally have a higher metabolism compared to humans (Sjogren et al., 2014).

### Conclusion

In vivo studies revealed a good preliminary indication of the of the polymeric hydrogel pharmaceutical dosage form's electro-responsive capabilities, ultimately facilitating the immediate release of the entrapped drug into the tissues and will significantly desensitize the patient to chronic pain whilst prohibiting any adverse effects. Indomethacin levels in the plasma were 6.29×10⁻⁹ to 6.76×10⁻⁷ μg/mL greater than that obtained by the conventional IV administration. In addition, the drug delivery system was well tolerated, showing no signs of inflammation. A Level A correlation as determined by IVIVC correlation further provided evidence on the feasibility of the polymeric hydrogel pharmaceutical dosage form and the transdermal system in use. Ultimately, the study served as determining the feasibility of such a prototype dosage form and transdermal system for expanding it to human trials.

The polymeric hydrogel pharmaceutical dosage form according to the invention provides an electro-responsive dosage form for the delivery of a drug to a target site on a human or animal, preferably the target site being the dermis of the human or animal.

The Applicant is not aware of any hydrogel having both polyethyleneimine (PEI) and 1-vinylimidazole (1VA). There is no prior art that the Applicant is aware of that would motivate any combination of polyethyleneimine (PEI) and 1-vinylimidazole (1VA) to form a hydrogel, let alone a polymeric hydrogel pharmaceutical dosage form comprising polyethyleneimine (PEI), 1-vinylimidazole (1VA), polyvinyl alcohol (PVA) and polyacrylic acid (PAA).

The polymeric hydrogel pharmaceutical dosage form according to the invention at least ameliorates the disadvantages in the prior art, and provides for a dosage form to be utilized in a method of treating chronic pain wherein a patient can readily control the increase or decrease of the release rate of analgesic being released from the dosage form in order to effectively manage chronic pain. The physical structure of the novel and inventive polymeric hydrogel pharmaceutical dosage form is not compromised through continued exposure to electrical stimuli and remains effective in use, therein providing for an effective means to manage chronic pain.

While the invention has been described in detail with respect to specific embodiments thereof, it will be appreciated that those skilled in the art, upon attaining an understanding of the foregoing may readily conceive of alterations to, variations of and equivalents to these embodiments. Accordingly, the scope of the present invention should be assessed as that of the claims and any equivalents thereto.

### REFERENCES

Hoff J., 2000. Methods of blood collection in the mouse. Lab Animal 29(10), 47-53
Lawson, P. T., 2000. Laboratory Animal Technician Training Manual, American Association for Laboratory Animal Science.
Prabu, S. L., Suriyaprakash T. N. K., 2012. Extraction of Drug from the Biological Matrix: A Review, Applied Biological Engineering—Principles and Practice, Dr. Ganesh R. Naik (Ed.), ISBN: 978-953-51-0412-4, InTech, DOI: 10.5772/32455. Available from: http://www.intechopen.com/books/applied-biological-engineering-principles-and-practice/extraction-of-the-drug-from-the-biological-matrix
Raveendran, R., Heybroek, W. M., Caulfield, M., Abrams, S. M., Wrigley, P. F., Slevin, M., Turner, P., 1992. Protein binding of indomethacin, methotrexate and morphine in patients with cancer. International Journal of Clinical Pharmacology Research 12, 117-122.
Shimamura, T., Tairabune, T., Kogo, T., Ueda, H., Numajiri, S., Kobayashi, D., Morimot, Y., 2004. Investigation of the Release Test Method for the Topical Application of Pharmaceutical Preparations: Release Test of Cataplasm Including Nonsteroidal Anti-inflammatory Drugs Using Artificial Sweat. Chemical and Pharmaceutical Bulletin, 52(2), 167-171.
Simon, G. A., Maibach, H. I., 1998. Relevance of hairless mouse as an experimental model of percutaneous penetration in man. Skin Pharmacology and Applied Skin Physiology 11, 80-86.
Sjögren, E., Abrahamsson, B., Augustijns, P., Becker, D., Bolger, M. B., Brewster, M., Brouwers, J., Flanagan, T., Harwood, M., Heinen, C, Holm, R., Juretschke, H-P., Kubbinga, M., Lindahl, A., Lukacova, V., Minister, U., Neuhoff, S., Nguyen, M. A., Peer, A. v., Reppas, C, Hodjegan, A. R., Tannergren, C, Weitschies, W., Wilson, C, Zane, P., Lennernas, H., Langguth, P., 2014. In vivo methods for drug absorption—Comparative physiologies, model selection, correlations with in vitro methods (IVIVC), and applications for formulation/API/excipient characterization including food effects, European Journal of Pharmaceutical Sciences, doi: http://dx.doi.Org/10.1016/j.ejps.2014.02.010
Almousa, A. A., Ikeda, R., Wada, M., Kuroda, N., Hanajirib, R., Nakashima, K., (2011). HPLC-UV method development for fentanyl determination in rat plasma and its application to elucidate pharmacokinetic behavior after i.p. administration to rats. Journal of Chromatography B, 879, 2941-2944.
Anoopkumar-Dukie S., Lack B., Mcphail K., Nyokong T., Lambat Z., Maharaj D., Daya S., (2003). Indomethacin reduces lipid peroxidation in rat brain homogenate by binding Fe²⁺. Metabolic Brain Disease, 18(1), 1-9.
Bajpai, A. K., Bajpai, J., Soni, S. N., (2008). Preparation and characterization of electrically conductive composites of poly(vinyl alcohol)-g-poly(acrylic acid) hydrogels impregnated with polyaniline (PANI). eXPRESS Polymer Letters 2, 26-39.
Banga, A. K., Chien, Y. W., (1988). Iontophoretic delivery of drugs: fundamentals, developments and biomedical applications. Journal of Controlled Release, 7, 1-24.
Belma, I., (2000). Swelling Behavior of Acrylamide-2-Hydroxyethyl Methacrylate Hydrogels. Turkish Journal of Chemistry, 24, 147-156.
Chen, C. H., LaRue, J. C., Nelson, R. D., Kulinsky, L., Madou, M. J., (2012). Electrical Conductivity of Polymer Blends of Poly (3, 4-ethylenedioxythiophene): Poly(styrenesulfonate): N-Methyl-2-pyrrolidinone and Polyvinyl Alcohol. Journal of Applied Polymer Science, 000, 000-000.
Choonara, Y. E., Pillay, V., Ndesendo, V. M. K., du Toit, L. C., Kumar, P., Khan, R. A., Murphy, C. S., Jarvis, D-L., 2011. Design of Polymeric Nanoparticles by Synthetic Wet Chemical Processing Strategies for the Sustained Delivery of Anti-Tuberculosis Drugs. Colloids and Surfaces B: Biointerfaces 87, 243-254.
Escobar-Chávez, J. J., Bonilla-Martinez, D., Villegas-Gonzalez, M. A., Molina-Trinidad, E., Casas-Alancaster, N., Revilla-Vazquez, A. L., (2011). Microneedles: A Valuable Physical Enhancer to Increase Transdermal Drug Delivery. The Journal of Clinical Pharmacology, 51, 964-977.
Ferrero, C, Massuelle, D., Doelker, E., (2010). Towards elucidation of the drug release of the drug release mechanism from compressed hydrophilic matrices made of cellulose ethers. II. Evaluation of possible swelling-controlled drug release mechanism using dimensionless analysis. Journal of Controlled Release, 141, 223-233.
Forster, A., Hempenstall, J., Rades, T., (2001). Characterization of glass solutions of poorly water soluble drugs produced by melt extrusion with hydrophilic amorphous polymers. Journal of Pharmacy and Pharmacology, 53, 303-315.
Frank, D. H., Roe, D. J., Sherry Chow, H-H., Guillen, J. M., Choquette, K., Gracie D., Francis, J., Fish, A., Alberts, D. S., 2004. Effects of a High-Selenium Yeast Supplement on Celecoxib Plasma Levels A Randomized Phase II Trial. Cancer Epidemiology, Biomarkers & Prevention, 13 299-303.
Frutos, G, Prior-Cabanillas, A., Paris, R., Quijada-Garrido, I., (2010). A novel controlled drug delivery system based on pH-responsive hydrogels included in soft gelatin capsules. Acta Biomaterialia, doi:10.1016/j.actbio.2010.07.018.
Gudeman, L. F., Peppas, N. A., 1995. Preparation and characterization of pH sensitive, interpenetrating networks of poly (vinyl alcohol) and poly-(acrylic acid). Journal of Applied Polymer Science 55, 919-928.
Guiseppi-Elie, A., (2010). Electroconductive hydrogels: Synthesis, characterization and biomedical applications. Biomaterials, 31, 2701-2716.
Hamidi M, Azadi A, Rafiei, P., (2008). Hydrogel nanoparticles in drug delivery. Advanced Drug Delivery Reviews, 60, 1638-1649.
Kamal A. H. M., Ahmed M., Wahed M. I. I., Amran S., Shaheen S., Rashid M., Anwar-Ul-Islam M., (2008). Development of indomethacin sustained release microcapsule using ethyl cellulose and hydroxyl propyl methyl cellulose phthalate by o/w emulsification. Dhaka University Journal of Pharmaceutical Science, 7(1), 83-88.
Kumar, P., Pillay, V., Choonara, Y. E., Modi, G., Naidoo, D., du Toit, L. C., 2011. In silico theoretical molecular modeling for Alzheimer's disease: The nicotine-curcumin paradigm in neuroprotection and neurotherapy. International Journal of Molecular Sciences 12, 694-724.
Kurkuri, M. D., Aminabhavf, T. M., 2004. Poly(vinyl alcohol) and poly(acrylic acid) sequential interpenetrating network pH-sensitive microspheres for the delivery of diclofenac sodium to the intestine. Journal of Controlled Release, 96(1), 9-20.
Lee, Y. M., Kim, S. H., Cho, C. H., 1996. Synthesis and swelling characteristics of pH and thermoresponsive interpenetrating polymer network hydrogel composed of poly(vinyl alcohol) and poly(acrylic acid). Journal of Applied Polymer Science, 62(2), 301-311.
Lee, J. W., Park, J., Prausnitz, M., 2008. Dissolving microneedles for transdermal drug delivery. Biomaterials, 29(13), 2133-2124.
Morales, M. E., Gallardo Lara V., Calpena, A. C., Domenech J, Ruiz, M. A., (2004). Comparative study of morphine diffusion from sustained release polymeric suspensions. Journal of Controlled Release, 95 75-81.
Morales, M. E., Lopez G., Gallardo V., Ruiz, M. A., (2011). Oral Suspensions of Morphine Hydrochloride for Controlled Release: Rheological Properties and Drug Release. Molecular Pharmaceutics, 8, 629-634.
Moya-Ortega, M. D., Alvarez-Lorenzo, C, Sigurdsson, H. H., Conchiero, A., Loftsson, T., (2010). γ-Cyclodextrin hydrogels and semi-interpenetrating networks for sustained delivery of dexamethasone. Carbohydrate Polymers, 80, 900-907.
O'Brien, T., Breivik, H., (2012). The impact of chronic pain-European patients' perspective over 12 months. Scandinavian Journal of Pain, 3, 23-29.
Park, K. Y., Jang, W. S., Lim, Y. Y., Ahn, J. H., Lee, S. J., Kim, C. W., Kim, S. E., Kim, B. J., Kim, M. N., 2013. Safety evaluation of stamp type digital microneedle devices in hairless mice. Annals of Dermatolgy, 25(1), 46-53.
Peng, L., Kim, N. H., Siddaramaiah., Lee, J. H., (2009). Swelling behaviour of polyacrylamide/laponite clay composite hydrogels: pH-sensitive property. Composites: Part B, 40, 275-283.
Peppas, N. A., Bures, P., Leobandung, W., Ichikawa, H., (2000). Hydrogels in pharmaceutical formulations. European Journal of Pharmaceutics and Biopharmaceutics, 50, 27-46.
Samui, A. B., Jayakumar, S., Jayalakshmi, C. G., Pandey, K., Sivaraman, P., (2007). Electroactive polymer gels based on epoxy resin. Smart Materials and Structures, 16, 237-242.
Shalvari, A., Qiang, L., Abdekhodaie, M. J., Xiao, Y. W., (2010). Novel modified starch-xanthan gum hydrogels for controlled drug delivery: Synthesis and Characterization. Carbohydrate Polymers, 79, 898-907.
Van Damme, P., Oosterhuis-Kafejaa, F., Van der Wielen, M., Almagor, Y., Sharon, O., Levin, Y., 2009. Safety and efficacy of a novel microneedle device for dose sparing intradermal influenza vaccination in healthy adults. Vaccine, 27 (3), 454-459.
Zarzycki, R., Modrzejewska, Z., Nawrotek, K., (2010). Drug Release from Hydrogel Matrices. Ecological Chemistry and Engineering S, 17 (2), 117-136.

{All references stated hold significance to the statements referenced}

1. A polymeric hydrogel pharmaceutical dosage form for drug delivery to a target site of a human or animal, the dosage form comprising:
polyethyleneimine (PEI) and 1-vinylimidazole (1VA) forming an electro responsive matrix,
wherein application of an electrical stimulus to the dosage form induces a first conformational change in the dosage form resulting in a release conformation which facilitates an increase in the release rate of the drug from the dosage form to the target site, and
wherein cessation of the electrical stimulus to the dosage form induces a second conformational change in the dosage form resulting in a drug containing conformation which facilitates a decrease in the release rate of the drug from the dosage form to the target site.

2. The dosage form according to claim 1, further comprising polyacrylic acid (PAA) and/or polyvinyl alcohol (PVA).

3. The dosage form according to claim 2, further comprising a crosslinking agent.

4. The dosage form according to claim 3, wherein the crosslinking agent is N,N′-methylenebisacrylamide.

5. The dosage form according to claim 3, further comprising a crosslinking initiator.

6. The dosage form according to claim 5, wherein the crosslinking initiator is potassium persulfate.

7. The dosage form according to claim 3, wherein the crosslinking agent crosslinks at least one or more of the following group: polyacrylamide (PAA), polyethyleneimine (PEI) polyvinyl alcohol (PVA) and 1-vinylimidazole (1VA).

8. The dosage form according claim 1, wherein the target site is the dermis of the human or animal.

9. The dosage form according to claim 1 further comprising a drug.

10. The dosage form according to claim 9, wherein the drug is an analgesic.

11. The dosage form according to claim 10, wherein the analgesic is at least one selected from the group: indomethacin, morphine, celecoxib and fenatyl chloride.

12. The dosage form according to claim 1, wherein the electrical stimulus is an electric current.

13. The dosage form according to claim 12, wherein the electric current is applied to the dosage form for a time period of from 0.1 seconds to 60 seconds.

14. The dosage form according to claim 13, wherein the electric current has a voltage of from 0.3 volts to 5 volts.

15-26. (canceled)

27. A method of manufacturing a polymeric hydrogel pharmaceutical dosage form for drug delivery to a target site of a human or animal, the method comprising the following step(s):
preparing a polyvinyl alcohol (PVA) solution to which polyethyleneimine (PEI) and 1-vinylimidazole (1VA) is added to form a first mixture; adding a drug, acrylic acid and a crosslinking agent to the first mixture; and
allowing a hydrogel to form which contains the drug and is responsive to electrical stimulus.

28. The method according to claim 27, wherein the crosslinking agent is N,N′-methylenebisacrylamide.

29. The method according to claim 28, further comprising the step of adding a crosslinking initiator to the second solution.

30. The method according to claim 29, wherein the crosslinking initiator is potassium persulfate.
